(19) United States
(12) Patent Application Publication
(10) Pub. No.: US 2025/0009403 A1
(43) Pub. Date: Jan. 9, 2025

(54) ELECTRODES WITH ARC MITIGATION

(71) Applicant: Pulse Biosciences, Inc., Hayward, CA (US)

(72) Inventors: Cameron D. HINMAN, Thurmond, NC (US); David J. DANITZ, San Jose, CA (US); Kevin L. MOSS, Lathrop, CA (US)

(21) Appl. No.: 18/713,620

(22) PCT Filed: Nov. 23, 2022
(86) PCT No.: PCT/US2022/050972

§ 371 Date: May 24, 2024

Related U.S. Application Data
(60) Provisional application No. 63/284,626, filed on Nov. 30, 2021.

(51) Int. Cl. A61B 18/00

(52) U.S. Cl. CPC A61B 18/00; A61B 2018/00077; A61B 2018/00083; A61B 2018/00136; A61B 2018/00184; A61B 2018/00291; A61B 2018/00613

(57) ABSTRACT

Methods and apparatuses for applying electrical energy to a target tissue, including sub-microsecond pulsed electrical energy. These methods and apparatuses may prevent or limit arcing and/or maintain adequate contact during treatment while minimizing arcing. For example, described herein are electrodes that have a surface that is covered by an arc mitigating layer. The arc mitigating layer may be separated from the electrode surface by a gap region (e.g., air gap).

### CROSS REFERENCE TO RELATED APPLICATIONS

This patent application claims priority to U.S. Provisional Patent Application No. 63/284,626, titled “ELECTRODES WITH ARC MITIGATION,” filed on Nov. 30, 2021, which is herein incorporated by reference in its entirety.

### BACKGROUND

Very short and high field strength electric pulses have been described for electromanipulation of biological tissue and cells. For example, electric pulses may be used in treatment of tissue, including benign and malignant tumors, lesions and other conditions. Treatments with electric pulses, including higher electric field strengths and shorter electric pulses, may be useful in manipulating intracellular structures, such as nuclei and mitochondria. For example, sub-microsecond (e.g., nanosecond) high voltage pulse generators and treatment applicators have been proposed for biological, medical and cosmetic applications. However, such higher peak electric fields are more likely to arc between the electrodes.

Because of the extremely high therapeutic voltages, as well as the very fast pulse times, applicators for delivery of sub-microsecond electrical fields should ideally be configured so as to avoid arcing between the electrodes applying the energy. It may be extremely difficult to prevent arcing in situation in which the electrodes are surface electrodes that my not uniformly contact the target tissue, or in which gaps are present between the electrode surface and the tissue, and this problem is particularly apparent when larger electrodes are used. Described herein are methods and apparatuses that may address these issues and may generally provide enhanced treatment of tissue using sub-microsecond pulsed energy.

### SUMMARY OF THE DISCLOSURE

Described herein are methods and apparatuses (e.g., devices, systems, etc., including applicators for applying electrical energy) for applying electrical energy to a target tissue. In particular, described herein are methods and apparatuses for applying sub-microsecond pulsed electrical energy to target tissues that prevent or limit arcing and/or maintain adequate contact during treatment while minimizing arcing. For example, described herein are electrodes (e.g., surface electrodes) that include an electrode surface that is covered by an arc mitigating layer, e.g., a layer formed of a material having a conductivity that is less than the electrode surface. The arc mitigating layer may be flexible and/or may be separated from the electrode surface by a gap region. The gap region may, in some examples, be an air gap. Any of these electrodes may have an electrically conductive surface that is long, including greater than 5 mm long or greater, 7 mm long or greater, 10 mm long or greater, 12 mm long or greater, 15 mm long or greater, etc. Even with these exceptionally long electrodes, which may otherwise be difficult to maintain in uniform contact with the target tissue, the use of an arc mitigating covering as described herein may prevent arcing that may otherwise occur when applying sub-microsecond pulsing at high electrical field strengths. The electrode surface may also be referred to herein as the electrically conductive surface for convenience.

The methods and apparatuses described herein may include applicators having two or more electrodes that are configured to clamp tissue between the electrodes. Also described herein are applicators including two or more sets of electrodes that are configured to be driven against a target tissue that are on a paddle region that may be easily and effectively articulated to be driven against the target tissue. Any of the applicators described herein may include an electrically conductive surface that is covered by an arc mitigating layer, which may be spaced from the electrically conductive surface by a gap region (e.g., air gap) or in some configurations directly in contact with the electrically conductive surface. Alternatively or additionally, any of the electrically conductive surfaces described herein may include rounded edges (fillet(s)) that prevent charge accumulation.

For example, described herein are electrode apparatuses that limit arcing comprising: a first electrically insulative support; a first electrode supported by the first electrically insulative support, the first electrode comprising a first electrode surface; a first arc mitigating layer covering the first electrode surface; a second electrode supported by the first electrically insulative support or a second electrically insulative support, the second electrode comprising a second electrode surface; and a second arc mitigating layer covering the second electrode, wherein the first and second electrodes are configured to pass electrical pulses having an amplitude of at least 0.1 kV and a duration of less than 1000 nanoseconds therebetween without arcing.

Also described herein are electrode apparatuses (e.g., configured to limit arcing) comprising: a first electrically insulative support; a first electrode supported by the first electrically insulative support, the first electrode comprising a first electrode surface; and a second electrode supported by the first electrically insulative support or a second electrically insulative support, the second electrode comprising a second electrode surface, wherein the first electrode and the second electrode are configured to pass electrical pulses having an amplitude of at least 0.1 kV therebetween and wherein the first electrode comprises first rounded edges on or around the first electrode surface and the second electrode comprises second rounded edges on or around the second electrode surface, the first and the second rounded edges configured to reduce or eliminate arcing between the first electrode and the second electrode when passing the electrical pulses. The first electrode surface may be a tissue facing or tissue contacting surface and the second electrode surface may be a tissue facing or tissue contacting surface. The first rounded edges may be not only on or around the first tissue contacting surface but also on or around a first non-tissue contacting surface, and the second rounded edges may be not only on the second tissue contacting surface but also on or around a second non-tissue contacting surface. In some examples, the rounded edges/fillets are configured to reduce peak electric field by, for example, up to about 30%. In some examples, the electrode apparatus may comprise an elongate shaft, and the first jaw and the second jaw may be positioned at an angle (e.g., perpendicular) to the shaft and may have a curved or bent configuration. The first and the second jaw may be axially movable relative to each other. In some examples, optionally, the first electrode surface may be covered by a first arc mitigating layer and the second electrode surface may be covered by a second arc mitigating layer.

Any of these electrode apparatuses may be configured to limit arcing, and may include: a first electrically insulative support; a first surface electrode supported by the first electrically insulative support, the first surface electrode comprising a first electrode surface; and a second surface electrode supported by the first electrically insulative support or a second electrically insulative support, the second surface electrode comprising a second electrode surface, wherein the first and second surface electrodes are configured to pass electrical pulses having an amplitude of at least 0.1 kV therebetween and wherein the first electrode surface is covered by a first arc mitigating layer and the second electrode surface is covered by a second arc mitigating layer configured to reduce or eliminate arcing between the first and the second surface electrode when passing the electrical pulses.

Any of these electrode apparatuses may include an applicator for applying electrical energy as described herein. The electrode apparatuses described herein may include two or more electrodes (e.g., surface electrodes), such as three or more, four or more, five or more, etc. The electrode apparatus may be hand-held or robotically held, or both. The electrode apparatus may include an elongate body and/or a handle region. The electrode apparatus may be configured as a tip or tip region that is coupled (mechanically and/or electrically) to a reusable applicator handle. In some examples the electrode apparatus may be disposable or limited-use (e.g., for use in a single treatment session or with a single patient). In some examples the electrode apparatus may be reusable, e.g., sterilizable.

Any of these apparatuses may include one or more electrically insulative support. The electrically insulative support may be formed of an electrically insulating material on or within which the electrodes are held and/or formed. Any appropriate electrically insulative support material may be used, including polymeric materials. For example, the electrically insulative support may be formed of a polyethylene (PE), polyvinylchloride (PVC), polypropylene (PP) and polyamide (PA). The electrically insulative support may be formed into an applicator surface, such as a flat or curved surface, to assist in making contact with the tissue to be treated. In some cases the electrically insulative support may be formed into a paddle, jaw arm, or other structure holding and supporting the electrodes. In some examples the apparatus (e.g., applicator apparatus) may include two or more separate electrically insulative support, each supporting one or more (e.g., an array) of electrodes.

The electrodes described herein may comprise plate or surface electrodes that may be configured for applying sub-microsecond (e.g., nanosecond) energy to a surface of a tissue. In any of the apparatuses described herein the surface electrode may be configured to be held against the tissue to be treated (e.g., against a target region of the tissue). The surface electrode may be substantially flat or may include a flat, tissue-contacting surface. The surface electrodes described herein may have any appropriate shape, such as round, oval, hexagonal, square, rectangular, etc. In some examples the surface electrodes described herein are elongate, having a larger length than width. In some examples the electrodes described herein have a length that is greater than 2 times (2.5 times or greater, 3 times or greater, 4 times or greater, 5 times or greater, etc.) the width. For example, the electrodes may have a length that is 2 mm or longer, 3 mm or longer, 4 mm or longer, 5 mm or longer, 6 mm or longer, 7 mm or longer, 8 mm or longer, 9 mm or longer, 10 mm or longer, 11 mm or longer, 12 mm or longer, 13 mm or longer, 14 mm or longer, 15 mm or longer, etc.).

In any of the apparatuses described herein, the electrode may include an electrode surface. The electrode surface may be the face of the electrode that is configured to abut the tissue. The electrode surface may be substantially flat, or it may be curved or bent (e.g., slightly), or otherwise configured to be held against the target tissue surface. In some examples the electrode surface is formed of an electrical conductor, such as a conductive metal (e.g., silver, stainless steel, etc.). In some examples the electrode surface is formed of an electrically conductive polymer. In some examples the electrode surface is formed of a wire.

Any of the electrodes described herein may also include an arc mitigating layer covering the electrode surface. In some examples, the arc mitigating layer may be rigid and in other examples the arc mitigating layer may be formed of a flexible material that may be a membrane, coating, or the like. The arc mitigating layer may be formed of a material having a conductivity that has a conductivity (e.g., S/m at 20 degrees C.) that is less than the conductivity of the electrode surface and may be slightly less than, approximately equal to, or greater than the conductivity of the target tissue. For example, the conductivity of the arc mitigating layer may be between about 0.001 S/m and about 20 S/m (e.g., between about 0.01 S/m and about 10 S/m, between about 0.1 S/m and about 8 S/m, between about 0.1 S/m and about 5 S/m, between about 0.1 S/m and about 10 S/m, between about 0.1 S/m and about 4 S/m, between about 0.1 S/m and about 3 S/m, between about 0.1 S/m and about 2 S/m, etc., between about 0.1 S/m and about 1 S/m, between about 0.01 S/m and about 5 S/m, between about 0.01 S/m and about 3 S/m, etc.). For example, the arc mitigating layer may be formed of a polymeric material that is doped or otherwise treated to have a conductivity that is within the target range (e.g., between about 0.001 S/m and about 5 S/m). The polymeric material may be, but is not limited to, flexible polymeric materials such as silicones, or the like. The doping material (also referred to herein as filler material or filler) may be a conductive material such as, e.g., carbon (e.g., carbon nanotubes). For example, the arc mitigating layer may comprise a silicone polymer including a conductive filler (e.g., carbon nanotubes).

In general, the arc mitigating layer may cover the electrode surface. In some examples the arc mitigating layer is laminated directly onto the electrode surface. Alternatively or additionally, the arc mitigating layer may be attached over the electrically conductive layer with a gap or space therebetween. The gap or space may be an air gap, or a gap formed of a non-conductive fluid (e.g., air, pure water, oil, etc.). The non-conductive fluid may be compressible. The non-conductive fluid may be configured to be displaced into another portion of the electrode in fluid communication with the gap region between the arc mitigating layer and the electrode surface. In some example, the non-conductive fluid (e.g., air) may be driven out through an opening in the arc mitigating layer. The arc mitigating layer may be configured to have a relaxed configuration in which the arc mitigating layer is separated from the electrically conducive electrode surface by the gap region; when force (e.g., pressure, compression, vacuum, etc.) is applied to the hold or drive the target tissue against the electrode, the arc mitigating layer may be driven against the electrode surface allowing electrical energy (e.g., sub-microsecond pulsed electrical energy) to be applied.

For example, any of these apparatuses may include a gap region between the arc mitigating layer and the electrode surface in an unactuated state (when no force is being applied to drive the arc mitigating layer against the electrically conductive surface). When force is applied against the surface electrode (e.g., against the arc mitigating layer) the arc mitigating layer may be driven towards the electrically conductive surface collapsing the gap region. As mentioned, the gap region may be, for example, and air gap. Thus, the arc mitigating layer may be configured to be deflected against the electrically conductive surface when force is applied against the arc mitigating layer.

The gap region may be any appropriate spacing. For example, the gap region may have a width separating the arc mitigating layer and the electrode surface that is about 0.1 mm or greater, about 0.2 mm or greater, about 0.3 mm or greater, about 0.4 mm or greater, about 0.5 mm or greater, between about 0.1 mm and 5 mm, between about 0.2 mm and 5 mm, between about 0.3 mm and 5 mm, between about 0.4 mm and 5 mm, between about 0.5 mm and 5 mm, etc.

Any of the apparatuses described herein may include two or more electrodes, each including its own electrode (e.g., surface electrode) and arc mitigating layer. In practice the same arc mitigating layer lay be used to form more than one electrode but may be electrically isolated from each other (and may therefore be referred to herein as different arc mitigating layers, such as first arc mitigating layer, second arc mitigating layer, etc.). In some examples the arc mitigating layer may be secured to the insulative support. For example, the arc mitigating layer may be adhesively secured to the insulative support, tacked or welded to the insulative support, etc. The arc mitigating layers described herein may be referred to as arc mitigating membranes in some examples. The arc mitigating layers described herein may fully cover the underlying electrode surface.

In general the electrodes described herein are configured to pass electrical pulses having an amplitude of at least 0.1 kV and a duration of less than 1000 nanoseconds therebetween without arcing. For example, an apparatus as described herein may be configured to pass electrical pulses having an amplitude of about 1 kV or greater, about 2 kV or greater, about 3 kV or greater, about 5 kV or greater, about 6 kV or greater, about 7 kV or greater, about 8 kV or greater, about 9 kV or greater, about 10 KV or greater, between about 0.1 kV and 100 kV, between about 1 kV and about 100 kV, between about 3 kV and about 100 kV, between about 5 kV and about 100 kV, etc. The pulses may be sub-microsecond pulses (e.g., less than about 1000 ns, e.g., between about 1 ns and about 1000 ns, between about 1 ns and about 950 ns, between about 1 ns and about 900 ns, between about 5 ns an about 1000 ns, between about 5 ns and about 950 ns, between about 5 ns and about 900 ns, etc.).

Any of these apparatuses may be configured as clamping or grasping applicators. For example, any of these apparatuses may include a first electrode on a first jaw of the applicator and the second electrode is on a second jaw of the applicator. The first and second jaws may be configured to be open or closed relative to each other to secure a tissue to be treated therebetween. The jaws may be configured to open and close so that the first surface electrode and the second surface electrode remain parallel to each other (e.g., parallel opening jaws). The jaws may be configured to open in a scissor-like manner. The jaws may be configured so that one jaw moves relative to the other; for example, the first jaw may be configured to move axially relative to the second jaw (or vice versa).

The electrodes may be on the clamping surfaces of the jaws, such as the surfaces that face each other. Alternatively, the electrodes may be on lateral faces of the jaws, so that opening/closing the jaws increases or decreases the spacing between the electrodes but does not clamp the tissue between the electrodes.

In some examples the electrodes are on the same support structure, rather than on opposite jaws. The support structure may be configured to expand or contract to allow a user (or to automatically in some examples) adjust the spacing between the electrodes. In some examples, the apparatus may be configured to detect spacing between the electrodes and/or the jaws. For example, the apparatus may incorporate linear potentiometer. Alternatively or additionally, the first electrode and the second electrode may be separated by a fixed distance. In some examples the electrodes are arranged on a paddle structure that may be articulated to allow better contact with the tissue and/or to adjust the treatment region.

As mentioned, the first surface electrode and the second surface electrode may each comprise an elongate surface electrode, e.g., having a length that is greater than the width. For example, the electrode surface may be elongated, and may have a length that is greater than its width. In one example, the surface electrode(s), e.g., first electrode and/or the second surface electrode, may extend greater than 5 mm in length. In any of the examples that comprise an arc mitigating layer, the arc mitigating layer may extend over the electrode surface further than the electrode surface itself, or it may be the same size as the electrode surface.

Any of the electrodes described herein may include rounded edges/fillets on the electrode surface, which may also or further reduce the likelihood of arcing.

The electrodes (e.g., a first surface electrode and second surface electrode) may be formed of any appropriate conductive material. For example, the surface electrodes may be formed of stainless steel.

In any of these apparatuses, the arc mitigating layer(s), e.g., the first and second arc mitigating layers, may comprise a flexible membrane.

Any of these apparatuses may include one or more suction ports through the insulative support(s) configured to apply suction to draw a tissue against the electrode(s). For example, the apparatus may include one or more suction ports adjacent to the surface electrodes, including, but not limited to between the surface electrodes, underneath the surface electrodes, around the surface electrodes, etc. Thus, suction may be applied, e.g., from a distal end, though a suction channel passing through the applicator or in communication with the applicator, to ensure contact between the tissue and the surface electrodes. In some examples suction may be used to drive the arc mitigating layer against the electrode surface.

For example, described herein are electrode apparatuses that may limit or prevent arcing, even when used with large surface electrodes at high voltage, sub-microsecond pulsing. The electrode apparatus may include: a first electrically insulative support; a first surface electrode supported by the first electrically insulation support, the first surface electrode comprising a first electrode surface; a first flexible arc mitigating layer covering the first electrode surface, wherein the first flexible arc mitigating layer is separated from the first electrode surface by a first gap region; a second surface electrode supported by the first electrically insulative support or a second electrically insulative support, the second surface electrode comprising a second electrode surface; and a second flexible arc mitigating layer covering the second surface electrode, wherein the second flexible arc mitigating layer is separated from the second electrode surface by a second gap region, wherein the first and second electrodes are configured to pass electrical pulses having an amplitude of at least 0.1 kV and a duration of less than 1000 nanoseconds therebetween without arcing.

Also described herein are methods of using any of these apparatuses to treat a tissue. These methods may be methods specific to treat a particular indication, such as, but not limited to, methods of treating endometriosis, methods of treating vocal cord lesions (e.g., vocal cord polyps, nodules and/or cysts), methods of treating cardiac tissue (e.g., treating atrial fibrillation), or the like. For example, a method of applying a pulse electrical field energy to a target tissue may include: pressing a first surface electrode against the target tissue such that a first arc mitigating layer covering at least a portion of a first electrode surface is driven against the first electrode surface; pressing a second surface electrode against the target tissue such that a second arc mitigating layer covering at least a portion of a second electrode surface is driven against the second electrode surface; and applying a plurality of electrical pulses having an amplitude of greater than 0.1 kV and a duration of less than 1000 nanoseconds between the first and second surface electrodes to treat the target tissue while mitigating or preventing arcing between the first electrode surface and the second electrode surface.

In some examples of the methods, instead of the first arc mitigating layer and the second arc mitigating layer, the rounded edges are used on the first and the second electrode to treat the tissue while mitigating or preventing arcing between the first electrode and the second electrode. Any of these methods may include a first and second surface electrodes that are pressed against the target tissue by compressing the target tissue therebetween. For example, a first and second surface electrodes may be pressed against the target tissue by applying suction from an applicator holding the first and second surface electrodes to secure the first and second surface electrodes against the target tissue. In the examples with the arc mitigating layers, the first surface electrode may be pressed against the target tissue so that the first arc mitigating layer displaces a first air gap between the first arc mitigating layer and the first electrode surface. The second surface electrode may be pressed against the target tissue so that the second arc mitigating layer displaced a second air gap between the second arc mitigating layer and the second electrode surface.

Pressing the first surface electrode against the target tissue may comprise pressing against the first electrode surface so that rounded edges of the first electrode surface contact the target tissue.

The first arc mitigating layer may have an electrical conductance that is less than the electrical conductivity of the first electrode surface, as described herein.

Treating the tissue while mitigating or preventing arcing may comprise mitigating or preventing arcing between non-tissue contacting portions or regions of the first electrode surface and the second electrode surface.

The methods may be a method of treating endometriosis, as mentioned. For example, in some examples pressing the first surface electrode against the target tissue comprises pressing the first surface electrode against an endometrial tissue.

The methods may be a method of treating a vocal cord/vocal fold, as mentioned. For example, pressing the first surface electrode against the target tissue may comprise pressing the first surface electrode against a vocal cord tissue.

The methods may be a method of treating a cardiac tissue, as mentioned. For example, pressing the first surface electrode against the target tissue may comprise pressing the first surface electrode against a cardiac tissue. For example, in some implementations, the method of applying a pulse electrical field energy to a cardiac tissue is provided and may include: pressing a first electrode surface of a first electrode against a first location on the cardiac tissue, wherein the first electrode comprises first rounded edges on or around the first electrode surface; pressing a second electrode surface of a second electrode against a second location on the cardiac tissue, wherein the second electrode comprises second rounded edges on or around the second electrode surface, the first and the second rounded edges; and applying a plurality of electrical pulses having an amplitude of greater than 0.1 kV and a duration of less than 1000 nanoseconds between the first and second surface electrodes to treat the cardiac tissue while mitigating or preventing arcing between the first electrode and the second electrode.

A method of applying a pulse electrical field energy to a target tissue may include: pressing a first surface electrode against the target tissue, wherein the first surface electrode comprises a first electrically conductive surface separated from a first flexible arc mitigating layer by a first air gap, so that the first arc mitigating layer is driven against the first electrically conductive surface over at least a region of the first electrically conductive surface; pressing a second surface electrode against the target tissue, wherein the second surface electrode comprises a second electrically conductive surface separated from a second flexible arc mitigating layer by a second air gap, so that the second arc mitigating layer is driven against the second electrically conductive surface over at least a region of the second electrically conductive surface; and applying a plurality of electrical pulses having an amplitude of greater than 0.1 kV and a duration of less than 1000 nanoseconds between the first and second surface electrodes to treat the tissue without arcing between the first electrically conductive surface and the second electrically conductive surface.

A method of applying a pulse electrical field energy to a target tissue may include: pressing a first surface electrode against the target tissue such that a first arc mitigating layer covering at least a portion of the first electrode surface is driven against the first electrode surface; pressing a second surface electrode against the target tissue such that a second arc mitigating layer covering at least a portion of the second electrode surface is driven against the second electrode surface; and applying a plurality of electrical pulses having an amplitude of greater than 0.1 kV between the first and second surface electrodes to treat the target tissue while mitigating or preventing arcing between the first electrode surface and the second electrode surface.

The apparatuses described herein may be particularly well suited for laparoscopic indications. These apparatuses may be used to treat tissue surfaces within a body lumen. For example, these apparatuses may be used to laparoscopically treat endometriosis (such as target tissue of the female reproductive system tissue, e.g., the ovaries, fallopian tubes, ligaments that support the uterus (uterosacral ligaments), posterior cul-de-sac, i.e., the space between the uterus and rectum, anterior cul-de-sac, i.e., the space between the uterus and bladder, outer surface of the uterus, lining of the pelvic cavity, and in some cases the intestines, rectum, bladder, vagina, cervix, and/or vulva). The apparatuses described herein may be particularly well suited for treating the heart (for example, to perform a Maze procedure used to treat an irregular heart rhythm (atrial fibrillation or AF)), and or within the oral cavity, esophagus, etc.

For example, also described herein are apparatuses that include: an elongate body; a paddle region extending distally from the elongate body, the paddle region comprising a lateral side and two or more surface electrodes extending in parallel along the lateral side; an articulating region coupling the elongate body to the paddle region, wherein the articulating region is configured to articulate the paddle region in a plane extending through the elongate body; and a handle at a proximal end including a control configured to articulate the distal paddle through over 180 degrees (e.g., 190 degrees, 200 degrees, etc.).

Any of these apparatuses may include an elongate body. The elongate body may be rigid and may be straight or curved. In some examples the elongate body is sized for insertion into a body region (e.g., between about 6 inches and 24 inches in length, etc.) The elongate body may be configured to be hand-held, and/or inserted through an introducer or mount.

The distal paddle region may support the two or more surface electrodes and may include or be formed in part of the electrically insulative support. The paddle region may be oval shaped, and/or may have an atraumatically shaped distal end region. The paddle region may be configured to contact the surface of the tissue to be treated (the target tissue) on a lateral side. The lateral side may be a side that is perpendicular to the distal end of the apparatus. In some examples, in the relaxed (unarticulated configuration) the paddle region is configured to be in-line with the long axis of the elongate member.

The articulating region may be configured to be driven to move the paddle region, and therefore the surface electrodes, in a single plane. For example, the articulating region may comprise a hinged region or a series of hinges forming the region. The hinges may include one or more pinned joints or may be formed of a liming hinge configuration. Articulating region may be articulated by one or more tendons (e.g., wires, string, cords, etc.) that may be used to pull and/or push the distal end of the articulating region relative to the proximal end of the articulating region (e.g., the paddle region relative to the elongate housing). The articulating region may be biased in one direction, e.g., to preferentially bend in one direction (“up” or “down”) or to return to the unbent configuration. The articulating region may be configured to bend in a single plane so that the paddle region travels between, e.g., +/−45 degrees, 50 degrees, 60 degrees, 65 degrees, 70 degrees, 75 degrees, 80 degrees, 85 degrees, 90 degrees, 95 degrees, 100 degrees, 105 degrees, 120 degrees, etc. Thus, the apparatus may be configured to articulate the distal paddle through over 90 degrees, 100 degrees, 110 degrees, 120 degrees, 130 degrees, 140 degrees, 150 degrees, 160 degrees, 170 degrees, 180 degrees, 190 degrees, 200 degrees, 210 degrees, etc.). The articulating region may be configured as an articulation joint.

Any of these apparatuses may include a control to control articulation of the device, including articulating of the articulating region. The control may be on the handle. The control (“articulation control”) may be a slider, knob, puller, dial, etc. The control may be mechanical or electrical. Any of these apparatuses may include a lock and locking control for locking and maintaining the selected angle of articulation. For example, the apparatus may include an articulation control with a locking button, knob, etc. for holding (locking) the selected angle. The lock may be mechanical and may prevent further movement of the tendon or gearing configured to drive articulation of the articulation region. In some examples the lock may include a gear and pawl. The lock may include a friction lock for preventing or limiting movement of a tendon articulating the articulating region.

Any of these apparatuses may include one or more suction ports on the lateral side and configured to apply suction to secure the two or more surface electrodes to a tissue surface.

In some examples the paddle region may be relatively flat, having a flat or curved lateral surface on one side and an opposite side that is also flat or curved, separated by a width. The width may be relatively thin (e.g., between 1 mm and 10 mm, between 1 mm and 7 mm, between 1 mm and 6 mm, etc.). The opposite side (back side) of the lateral surface may be referred to a back of the paddle region. In some example, the back of the paddle region may comprise an electrical insulation. In some example, the back side may include a second set of surface electrodes. The apparatus may be configured to allow the user (or a controller, such as a robotic controller) select between applying energy from the set of surface electrodes on the lateral side of the paddle region or from the set of electrodes on the back side. Thus, the apparatus may be configured to multiplex between the first set of surface electrodes on the lateral side and the second set of surface electrodes on the back side. For example, the handle region may include a control (e.g., switch) for switching between the sets of electrodes. Thus, any of these apparatuses may include a second set of two or more surface electrodes on a back of the paddle region that is opposite from the lateral surface and a control configured to actuate either the two or more surface electrodes on the lateral surface or second set of two or more surface electrodes on the back of the paddle region.

In some examples, the lateral side is a flat lateral surface. Alternatively, the lateral side may be curved, e.g., slightly curved (having a radius of curvature that is greater than, e.g., 10 mm); the lateral side may be curved in an axis that is perpendicular to the long axis (the proximal to distal axis). Alternatively in some examples the paddle region and in particular the paddle region may be curved in the long axis.

Any of these apparatuses (including those shown in FIGS. 3-9) may be configured so that the spacing of the surface electrodes relative to each other may be adjusted. In some examples the paddle region may be configured to expand in width (e.g., in a direction perpendicular to the long axis) so that the paddle region (and in some examples the lateral side of the paddle region) expands laterally to increase spacing between the two or more surface electrodes. The apparatus may include a control, e.g., an expansion control on the handle configured to increase and decrease the spacing between the two or more surface electrodes. The expansion may be drive by a tendon (wire, cord, etc.) and may be biased in an expanded or unexpanded configuration. The apparatus may also include an expansion lock configured to secure the paddle region in a selected expansion state.

In general, the two or more surface electrodes may comprise three or more surface electrodes. The surface electrodes may be similar to those described above, for example, the surface electrodes may each include an electrode surface and an arc mitigating layer covering the electrode surface (e.g., a flexible arc mitigating layer). In some examples the flexible arc mitigating layer may be separated from the electrode surface by a gap region (e.g., an air gap). The two or more surface electrodes may have rounded edges.

The surface electrodes may extend in parallel along a distal-to-proximal length of the lateral surface, e.g., for 5 mm or more (e.g., for 6 mm or more, 7 mm or more, 8 mm or more, 9 mm or more, 10 mm or more, etc.).

In some examples, an apparatus may include: an elongate body; a paddle region extending distally from the elongate body, the paddle region comprising a lateral side and two or more elongate surface electrodes extending in parallel along the lateral side; an articulating region coupling the elongate body to the paddle region, wherein the articulating region is configured to articulate the paddle region in a plane extending through the elongate body; and a handle at a proximal end including a control configured to articulate the distal paddle through over 200 degrees.

Also described herein are methods of using these apparatuses, including (but not limited to) method of using these apparatuses for a laparoscopic procedure. For example, as mentioned above, these methods may be used to treat endometriosis, a respiratory (e.g., bronchial) indication, a cardiac tissue and/or an oral/nasal indication (e.g., vocal cord/vocal folds), etc. For example, a method of treating a tissue may include: inserting an apparatus including an elongate body into a subject's body; bending an articulating region at a distal end of the elongate body so that a paddle region at a distal end of the articulating region moves in a plane extending through the elongate body; placing a lateral side of the paddle region against a target tissue region so that two or more surface electrodes extending in parallel along the lateral side are in contact with the target tissue; and applying a plurality of electrical pulses having an amplitude of greater than 0.1 kV and a duration of less than 1000 nanoseconds between the two or more surface electrodes to treat the target tissue. The paddle region may move in the plane extending through the elongate body through more than 180 degrees.

Any of these methods may include repositioning the apparatus against another region of the target tissue and applying a second plurality of electrical pulses. Placing the lateral side of the paddle region against the target tissue may comprise pressing the surface electrodes, wherein each surface electrode comprises an electrically conductive surface that is covered by an arc mitigating layer, against the target tissue so that the arc mitigating layer of each surface electrode is driven against the electrically conductive surface of each surface electrode.

Driving the arc mitigating layer (which may be a flexible layer) of each surface electrode against the electrically conductive surface of each surface electrode may comprise displacing a gap region between the flexible arc mitigating layer of each surface electrode and the electrically conductive surface of each surface electrode. The gap region may comprise an air gap.

Placing the lateral side of the paddle region against the target tissue may comprise pressing the lateral side of the paddle region against an endometrial tissue. Placing the lateral side of the paddle region against the target tissue may comprise pressing the lateral side of the paddle region against a vocal cord tissue. Placing the lateral side of the paddle region against the target tissue may comprise pressing the lateral side of the paddle region against a cardiac tissue.

In general, the techniques and features (including, for example, the electrodes and apparatuses) described herein may be applied to or adapted for use with any of the apparatuses and methods described in PCT application PCTUS2021035146, titled “HIGH-VOLTAGE MINIMALLY INVASIVE APPLICATOR DEVICES FOR SUB-MICROSECOND PULSING”, filed Jun. 1, 2021, which is herein incorporated by reference in its entirety.

The apparatuses described herein may be used with any pulse generator, including pulse generators configured for sub-microsecond (e.g., nanosecond), high voltage pulse generation. Sub-microsecond (e.g., nanosecond), high voltage pulse generators appropriate for biological and medical applications may include U.S. Patent Application No. 2008/0231337, U.S. Patent Application No. 2010/0038971, and US2021/0187292. The entire content of these publications is incorporated herein by reference.

All of the methods and apparatuses described herein, in any combination of various disclosed features, are herein contemplated and can be used to achieve the benefits as described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A better understanding of the features and advantages of the methods and apparatuses described herein will be obtained by reference to the following detailed description that sets forth illustrative embodiments, and the accompanying drawings of which:

FIG. 1 illustrates one example of a system as described herein, including a pulse generator.

FIGS. 2A-2B show top and perspective (sectional) views, respectively of an example of a contact electrode as descried herein.

FIGS. 2C-2D show top and perspective (sectional) views, respectively of an example of a contact electrode including an arc mitigating layer as descried herein.

FIGS. 2E-2F show top and perspective (sectional) views, respectively of another example of a contact electrode including an arc mitigating layer as descried herein.

FIGS. 2G and 2H illustrate use of a contact electrode including an air gap between an electrode surface and an arc mitigating layer before (FIG. 2G) and after (FIG. 2H) contacting with a tissue.

FIGS. 3A-3E illustrate one example of an applicator apparatus including a paddle region as descried herein.

FIGS. 4A-4F illustrate an example of an applicator apparatus including an expandable paddle region as described herein.

FIGS. 5A-5C illustrate side (FIGS. 5A and 5B) and perspective views (FIG. 5C) of another example of an apparatus similar to that shown in FIGS. 4A-4F.

FIG. 6 is an example of an apparatus having expandable jaws with side facing electrodes.

FIGS. 7A and 7B show examples of alternative jaw geometries for an apparatus having expandable jaws.

FIGS. 8A-8D show another example of an applicator apparatus having a pair of clamping jaws.

FIGS. 9A-9B show further examples of an apparatus having expandable jaws.

FIG. 10A shows an example of a section through a contact electrode that may be used with the clamping applicator, for example, similar to that shown in FIG. 9A.

FIGS. 10B-10E show alternative contact electrode configurations as described herein.

FIG. 11 illustrates one example of a model of a clamping apparatus including a contact electrode having an arc mitigating cover layer.

FIGS. 12A-12B show the results of a simulation of electric field intensities using the model shown in FIG. 11.

FIGS. 13A-13B show the results of another simulation of electric field intensities using the model shown in FIG. 11.

FIGS. 14A-14B show the results of a further simulation of electric field intensities using the model shown in FIG. 11.

FIG. 15 illustrates another example of a model of a clamping apparatus including a contact electrode having an arc mitigating cover layer.

FIGS. 16A-16B show the results of a simulation of electric field intensities using the model shown in FIG. 15.

FIG. 17A-17B show the results of another simulation of electric field intensities using the model shown in FIG. 15.

FIGS. 18A-18B show the results of a further simulation of electric field intensities using the model shown in FIG. 15.

### DETAILED DESCRIPTION

Described herein are methods and apparatuses (e.g., devices, systems, etc.) for applying electrical energy to tissue. In particular, these methods and apparatuses may be particularly well suited for applying high electrical field, such as sub-microsecond pulses (e.g., nanosecond pulses). The application of high electrical field pulses may result in undesirable arcing, particularly when applying energy between electrodes that are not uniformly in contact with the target tissue. This may result in limiting the size of the electrodes, favoring shorter, smaller electrodes, but requiring additional treatment steps in order to treat larger tissue regions. The methods and apparatuses described herein are configured to prevent or minimize/limit arcing even when used with larger surface electrodes (e.g., having a length of 4 mm or longer, 5 mm or longer, 6 mm or longer, 7 mm or longer, 8 mm or longer, 9 mm or longer 10 mm or longer, etc.), and/or when used in uneven tissue regions. Moreover, the methods and apparatuses described herein allow to achieve transmural lesions along an entire desired length of the treated tissue.

Also described herein are applicators including surface electrodes that may clamp onto tissue to provide treatment using high electrical field, such as sub-microsecond pulsing. Some examples of the applicators described herein are configured to apply energy against a tissue surface using a paddle region that includes surface electrodes. Any of these apparatuses may be configured to be used laparoscopically or in other minimally invasive applications and procedures. These apparatuses may also be configured to prevent arcing between the surface electrodes, including surface electrodes on different jaws, even where a portion of the surface electrode(s) may be exposed (e.g., not clamped on tissue or pressed against the tissue) to blood or air.

Although many of the examples described herein are described for the use of high field strength, sub-microsecond pulsing, any of these apparatuses and methods may be adapted for use with other forms of electrical energy

Described herein are apparatuses including instruments and devices, applicators, applicator tools, tips for applicators, etc. that may be configured as a laparoscopic device, a catheter, etc. or configured to be introduced or used through a lumen of a laparoscopic device, an endoscope or a catheter. Also described herein are methods for the treatment of a patient that may use any of these apparatuses to more effectively apply therapeutic energy, including but not limited to short, high field strength electric pulses, while minimizing or avoiding the risk of arcing or otherwise harming the tissue. These applicators may be used for minimally invasive procedures, and may be particularly well suited, for example, for treatments of various lesions, conditions, disorders and diseases, as stated in more detail below. These applications may be also particularly well suited for use with various fully and partially automated systems, such as robotic systems. In particular, the apparatuses described herein may be configured as apparatuses (e.g., laparoscopic apparatuses) that can be used with a variety of different generator systems, as will be described in greater detail herein.

Thus, the apparatuses described herein may be configured for manual or automated (e.g., robotic-assisted) control. In some examples these apparatuses may be integrated into systems that are configured to be mounted onto or coupled to a movable (e.g., robotic) arm of a robotic system, such as robotic medical treatment system or robotic surgical system. It should be understood that such surgical systems are intended to cover any robotic medical treatment system (including for cosmetic applications) and may include robotic systems having guidance. In some examples instruments can be guided and controlled by the robotic system during a surgical procedure. For example, the devices described herein may be used through one or more operating channels of a robotic system.

The apparatuses described herein may include elongate applicator tools that may be manipulated proximally (automatically or manually) to articulate the distal end region (also referred to as tip), including adjusting the opening/closing of the jaws in embodiments including jaws, adjusting and/or controlling the angle of the distal end region (e.g. paddle region) and/or the spacing of two or more surface electrodes at the distal end region. In some examples an applicator, as described herein, may include a proximal handle portion, an elongate body and a distal end region including two or more electrodes. The proximal handle may include one or more controls for manipulating the distal end region of the applicator tool, including articulating the distal end region to change the angle of the distal tip region relative to the elongate body, the rotational position of the distal end region relative to the elongate body, etc. One or more controls (including automatic controls) may also adjust the distance between pairs or sets of electrodes (e.g., cathode and anode) or sets of surface electrodes (cathodes and anodes).

The applicators may be referred to as elongate applicator tools. The elongate body portion may be rigid, bendable or flexible. In some examples the elongate body portion may be a catheter or catheter body.

According to one aspect, apparatuses described herein comprise medical devices and instruments for use in minimally invasive procedures that are introduced through a natural orifice (e.g. mouth, anus, etc.) or through a small incision and may be operated with additional tools, such as obturators, cameras, forceps, graspers, etc. In some examples, the apparatuses described herein may be used through a working channel of an endoscope. In some examples, these apparatuses may be configured as a catheter or including an elongate catheter body. In any of the apparatuses or systems described herein the elongate applicators may be configured as a laparoscope (and may be referred to herein as a laparoscope, a laparoscope apparatus, or a laparoscopic instrument). As used herein a laparoscope may, but does not necessarily have to, include one or more visualization components (e.g., a fiber optic, camera, lenses, filters, etc.). Thus, any of the apparatuses described herein may be configured as a scope. These apparatuses may be configured safely and reliably to deliver microsecond, nanosecond, picosecond, etc. pulses, and may include an electric field with a pulse width of between 0.1 nanoseconds (ns) and less than 1000 nanoseconds, or shorter, such as 1 picosecond, which may be referred to as sub-microsecond pulsed electric field. This pulsed energy may have high peak voltages, such as 0.5 to 5 kilovolts per centimeter (kV/cm), 10 kV/cm, 20 kV/cm, 100 kV/cm or higher. Treatment of biological cells may use a multitude of periodic pulses at a frequency ranging from 0.1 Hz to 10,000 Hz, and may trigger regulated cell death, for example, in the diseased tissue or abnormal growth, such as cancerous, precancerous or benign tumors. Selective treatment of such tumors, lesions or other unwanted growth with high voltage, sub-microsecond pulsed energy can induce regulated cell death within the treated cells without substantially affecting normal cells in the surrounding tissue due to its non-thermal nature. A subject may be a patient (human or non-human, including animals). A user may operate the apparatuses described herein on a subject. The user may be a physician (doctor, surgeon, etc.), medical technician, nurse, or other care provider.

Thus, the application of high voltage, fast electrical pulses may include applying a train of electrical pulses having a pulse width, for example, of between 0.1 nanoseconds (ns) and 1000 nanoseconds. Applying high voltage, fast electrical pulses may include applying a train of sub-microsecond electrical pulses having peak voltages of between, for example, 1 kilovolt per centimeter (kV/cm) and 500 kV/cm. Applying high voltage, fast electrical pulses may include applying a train of sub-microsecond electrical pulses at a frequency, for example, of between 0.1 Hz to 10,000 Hz.

For example, described herein are apparatuses for treating tissue. Any appropriate tissue may be treated, including tissue that can fit, for example, between the jaws of a treatment applicator. Some examples of the tissue that may be treated with the apparatuses and methods of the present disclosure include one or more organs (e.g., pharynx, esophagus, stomach, small intestine, large intestine, liver, gallbladder, mesentery, pancreas, larynx, trachea, bronchia, lungs, diaphragm, kidney, bladder, urethra, ovaries, fallopian tubes, uterus, vagina, testes, epididymis, vas deferens, prostate, bulbourethral glands, pituitary gland, pineal gland, thyroid gland, adrenal glands, heart, arteries, veins (e.g. pulmonary veins), lymph nodes, lymphatic vessel, spleen, thymus, skin, eyelids, lips, tongue, ear, nose, vocal cords, etc.) In some examples the apparatuses and methods described herein may be used to treat one or more of these tissues, as part of a minimally invasive therapy. In some examples the therapy may be for treatment of cancer, cardiac conditions, or endometriosis, just to name a few. In some examples the methods and apparatuses described herein may be used to treat a tumor or tumors, including cancerous, precancerous, benign or non-malignant tumors, lesions or growths.

Any of these apparatuses may be used with a pulse generator. For example, described herein are systems for treating tissue that may include: an elongate applicator tool as described herein (e.g., an elongate body, including in some examples having a distal end region that articulates, the elongate body comprising a set of electrodes at its distal end region); and a pulse generator configured to generate a plurality of electrical pulses, such as those having amplitude of at least 0.1 kV and a duration of less than 1000 nanoseconds. The system may include a connector, e.g., a high voltage connector adapted to couple the elongate applicator tool to the pulse generator, the pulse generator comprising a port configured to connect to the high voltage connector.

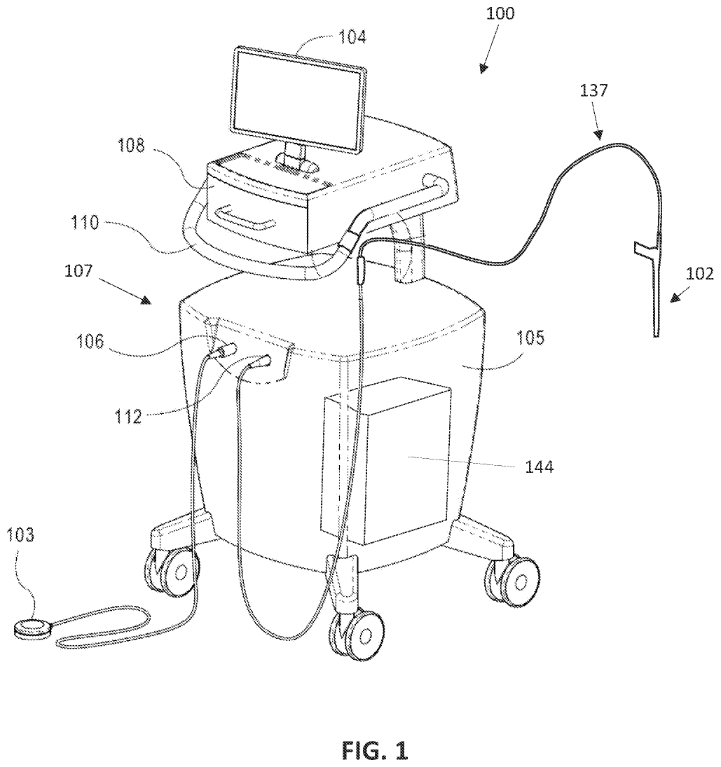

FIG. 1 illustrates one example of a system 100 (also referred to herein as a high voltage system or a sub-microsecond generation system) for delivering high voltage, fast pulses of electrical energy that may include an elongate applicator 102 (shown schematically), a pulse generator 107, footswitch 103, and user interface 104. The system 100 may provide the high voltage electrical energy pules to treat tissues, including tissues of one or more organs (e.g., pharynx, esophagus, stomach, small intestine, large intestine, liver, gallbladder, mesentery, pancreas, larynx, trachea, bronchia, lungs, diaphragm, kidney, bladder, urethra, ovaries, fallopian tubes, uterus, vagina, testes, epididymis, vas deferens, prostate, bulbourethral glands, pituitary gland, pineal gland, thyroid gland, adrenal glands, heart, arteries, veins, lymph nodes, lymphatic vessel, spleen, thymus, skin, eyelids, lips, tongue, ear, nose, vocal cords, etc.) In some examples the apparatuses and methods described herein may be used to treat one or more of these tissues, as part of a minimally invasive therapy. As stated above, in some examples the methods and apparatuses described herein may be used to treat a tumor or tumors, including cancerous, precancerous, benign or non-malignant tumors, lesions or growths. In some other examples, the methods and apparatuses described herein may be used to treat any feasible tissue or cell.

Footswitch 103 is connected to housing 105 (which may enclose the electronic components) through a cable and connector 106. The elongate applicator tool 102 may include electrodes and is connected to housing 105 and the electronic components therein through a cable 137 and high voltage connector 112. The high voltage system 100 may also include a handle 110 and storage drawer 108. The system 100 may also include a holder (e.g., holster, carrier, etc.) (not shown) which may be configured to hold the elongate applicator tool 102. The system 100 may include a controller 144 (shown schematically in FIG. 1), which may send signals to pulse control elements within system 100 or otherwise control operation of the pulse generator. The controller 144 may include one or more processors and may be coupled to the pulse generator either directly or indirectly. The controller may receive inputs from the one or more inputs and may provide output to the one or more outputs (e.g., monitors/touchscreens/interface, etc.). The controller 144 may be a microcontroller. The controller may include control circuitry and may include or be coupled with a memory, communications (e.g., wireless and/or wired) circuitry, etc.

A human operator may select a number of pulses, amplitude, pulse duration, and frequency information, for example by inputting such parameters into a numeric keypad or a touch screen of interface 104. In some examples, the pulse width can be varied. A controller 144 may send signals to pulse control elements within system 100. In some examples, fiber optic cables are used which allow control signaling while also electrically isolating the contents of the metal cabinet with sub-microsecond pulse generation system 100, e.g., the high voltage circuit, from the outside. In order to further electrically isolate the system, system 100 may be battery powered instead of being powered from a wall outlet.

The elongate applicator tool 102 may be hand-held (e.g., by a user) or it can be affixed to a movable arm of a robotic system, and its operation may be at least partially automated or fully automated, including computer-controlled.

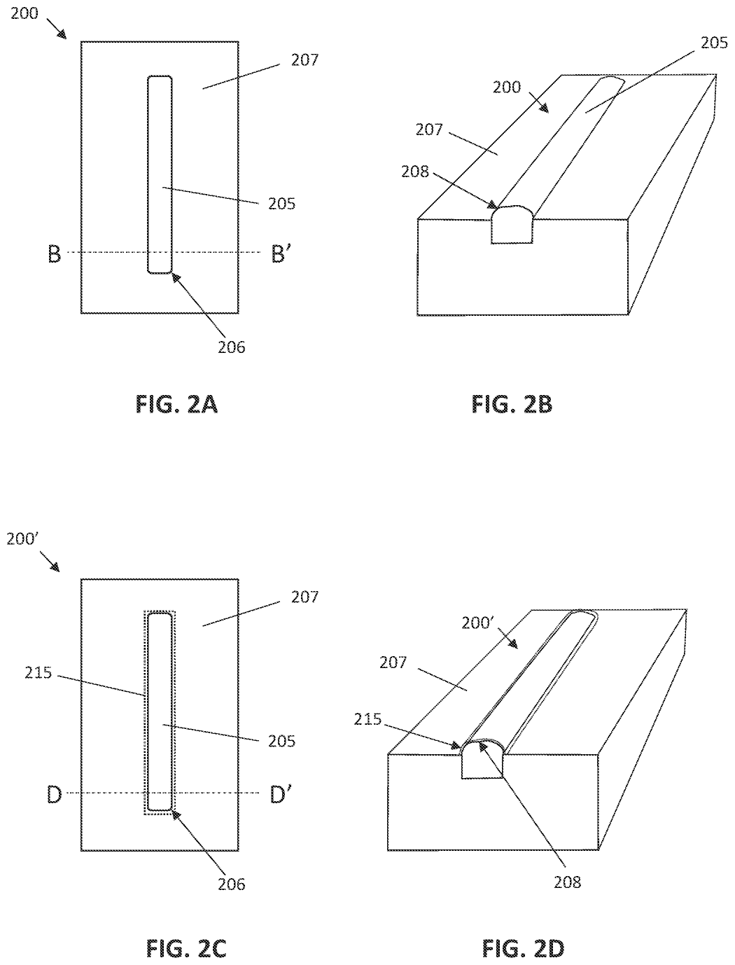

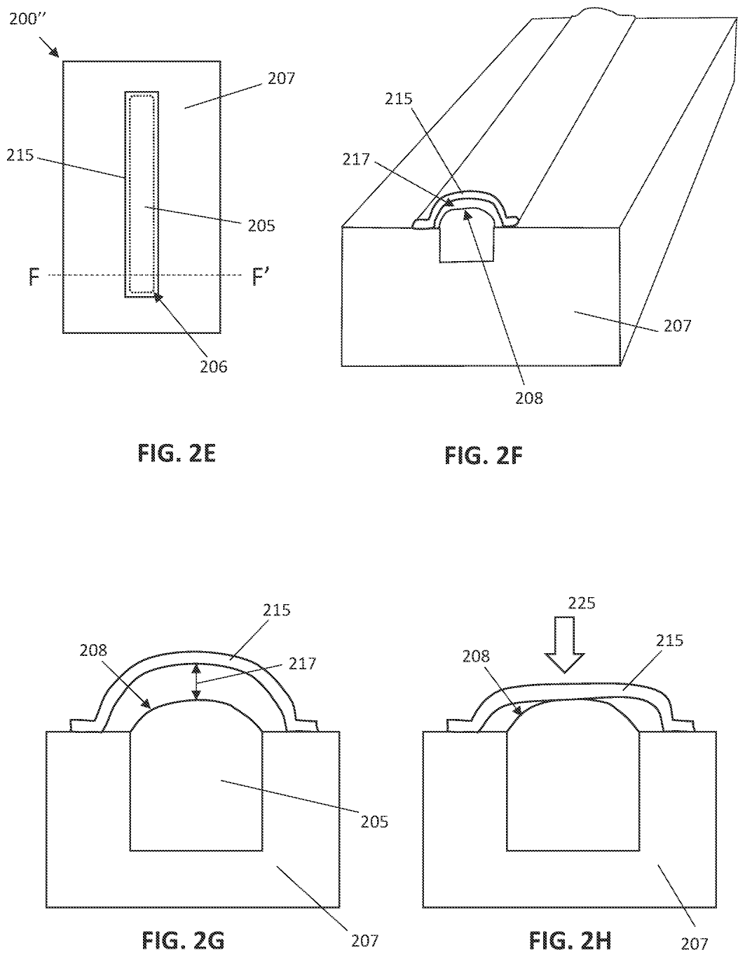

FIGS. 2A-2H illustrate examples of non-penetrating electrodes, such as surface or plate electrodes, that may be used with any of the apparatuses described herein. For example, FIGS. 2A and 2B show an example of a surface electrode assembly 200 that comprises an electrically insulative support 207. The electrically insulative support may be formed of any insulating material (e.g., polymeric material such as PE, PVC, PP and/or PA). As seen in FIG. 2B the electrode 205 includes an exposed (e.g., top) electrode surface 208 that is curved so that it does not present any sharp or abrupt edges. In this example, all of the edges of the electrode surface are rounded 206 as shown in FIG. 2A (e.g., include fillets) that have a radius of curvature, for example, of about 1 mm or greater. FIG. 2A shows a top view, while FIG. 2B shows a perspective view through a section (at B-B′). The electrode 205 may be formed of any appropriate electrically conductive material and may be coupled (e.g., within the support 207) to an electrical connector (not visible in FIGS. 2A-2B) for coupling to a pulse generator.

FIGS. 2C and 2D illustrate another example of a surface electrode assembly 200′ that is similar to that shown in FIGS. 2A-2B and includes an electrically insulating support 207 supporting an electrode 205, and also includes an arc mitigating layer 215 covering an exposed (e.g. top) electrically conductive surface of the electrode 205. The arc mitigating layer 215 may be formed of a material having an electrical conductivity that is less than the electrical conductivity of the electrode surface and preferably approximately (within 0.1 times to 10 times) the conductivity of the tissue to be treated. The arc mitigating layer may also be referred to as an intermediate conductivity layer or intermediate conductivity cover, as it may have an electrical conductivity that is intermediate to that of the electrode 205 and the insulative support 207. In some examples the arc mitigating layer may have an electrical conductivity that is one or two orders of magnitude less than or more than the electrical conductivity of the target tissue which it will contact in use. For example, the electrical conductivity of the arc mitigating layer may be between about 0.01 times and 20 times (e.g., between 0.1 times and 10 times, between 0.1 times and 5 times, etc.) the electrical conductivity of the tissue (e.g., skin, heart tissue, female reproductive tract tissue, etc.) to which it is to be applied. In some examples the arc mitigating layer may have an electrical conductivity (a) that is between about 0.01 siemens per meter (S/m) and about 20 S/m (e.g., between about 0.01 S/m and 10 S/m, between about 0.01 S/m and 5 S/m, between about 0.1 S/m and 20 S/m, between about 0.1 S/m and 10 S/m, between about 0.1 S/m and 5 S/m, between about 0.1 S/m and 1 S/m etc.).

The arc mitigating layer may be any appropriate thickness, such as, e.g., between 0.01 mm thick and 10 mm thick, between 0.01 mm thick and 5 mm thick, between about 0.01 mm thick and 3 mm thick, between about 0.1 mm thick and 10 mm thick, between about 0.1 mm thick and 5 mm thick, between about 0.1 mm thick and 3 mm thick, etc. The arc mitigating layer may be formed of a polymeric material that is biocompatible. In some examples, particularly (but not exclusively) when the electrode assembly includes a gap region (e.g., air gap) between the arc mitigating layer and the electrode surface, the arc mitigating layer may be formed of a material that is flexible. The arc mitigating layer may be formed of a polymeric material, such as silicone, that has been doped or otherwise treated to include a material to provide a conductance within the indicated range, such as carbon nanotubules. For example, in some variations the arc mitigating layer is formed of a silicone including carbon black or other conductive material so that it has an electrical conductivity that is within the ranges indicated above. In some examples the arc mitigating layer may be rigid and may be formed of a polymeric material that is not flexible, particularly when applied as a cover directly against the electrode surface.

In the surface electrode assembly 200′ shown in FIGS. 2C and 2D the arc mitigating layer 215 is applied against the exposed electrode surface 208 of the electrode 205. In some examples the arc mitigating layer may be separated from the exposed electrode surface by a gap region, as shown in FIGS. 2E-2H. In FIGS. 2E-2H the arc mitigating layer 215 is applied over the open or exposed electrode surface 208 of the electrode 205 spaced by a gap region 217. The gap region may be any appropriate distance and may generally include a fluid (e.g., air) that is insulating.

In use, the assembly 200″ with a plate or surface electrode 205 and electrically insulative (e.g. electrically insulating) support 207 shown in FIGS. 2E and 2F may be applied to the tissue and the force 225 of the application may drive the arc mitigating layer 215 across the gap region 217 to contact the exposed electrode surface, as shown in FIG. 2H, in which force 225 is applied against the arc mitigating layer 215. Removing the force may allow the arc mitigating layer 215 to restore the gap region 217, as shown in FIG. 2G.

Each of the examples shown in FIGS. 2A-2B, 2C-2D and 2E-2H may reduce or prevent arcing when the surface electrode is used as part of an apparatus as described herein. For example, the use of rounded edges (e.g., 206) may prevent or at least minimize arcing. The application of an arc mitigating layer 215 may also minimize or prevent arcing even in regions where the tissue is not uniformly contacting the surface electrode. Similarly, the surface electrode shown in FIGS. 2E-2F may further limit or prevent arcing when the surface electrode is used as part of an apparatus, as described in greater detail below.

The rectangular body of the electrode 205 underlying the electrode surface 208 shown in FIGS. 2A-2H may be formed of the same electrically conductive material (e.g., conductive metal, such as a stainless steel) as the electrode surface 208, or it may be formed of a separate conductor. Any of the apparatuses described herein may be configured to include a surface electrode as described and illustrated in FIGS. 2A-2H.

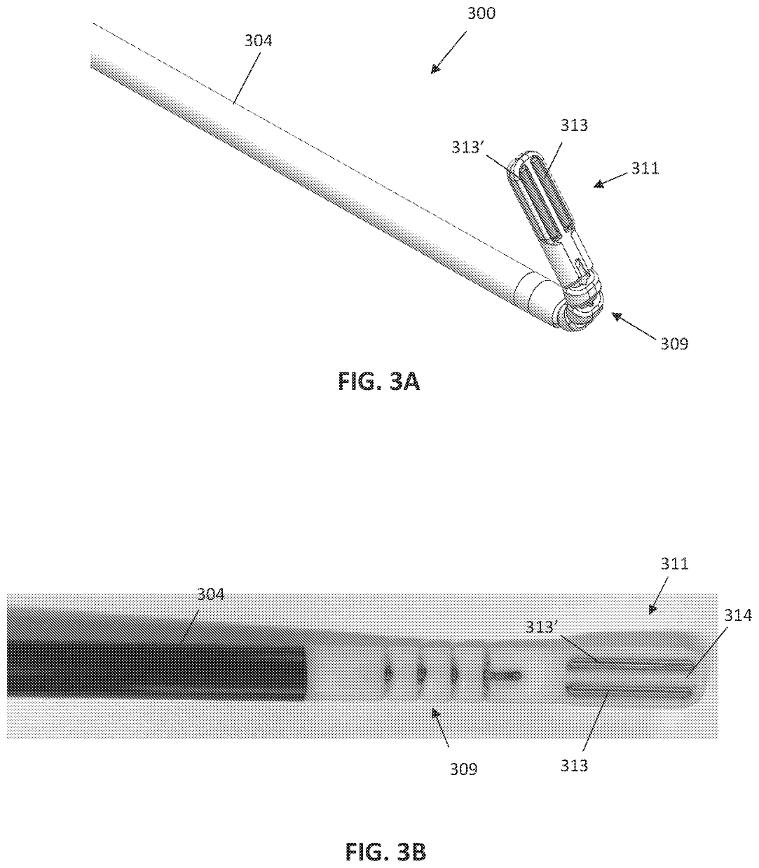

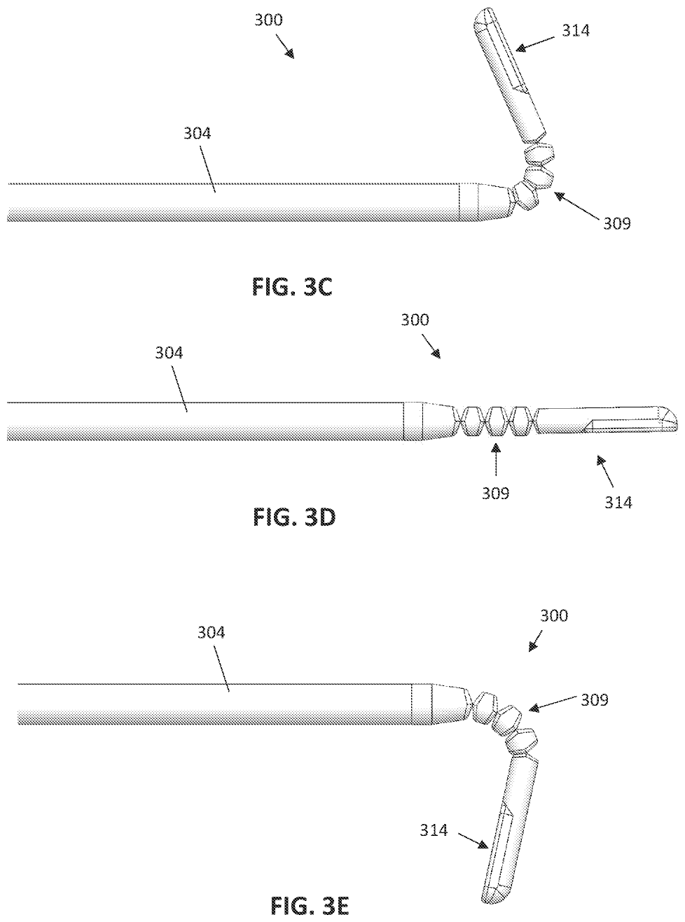

According to another aspect of the present disclosure, FIGS. 3A-3E illustrate one example of an apparatus configured for laparoscopic use that includes a distal paddle region on which two surface electrodes are included. This apparatus 300 includes an elongate body 304 that may extend proximally, for example, to a handle or to a connection to a movable arm (not shown). A paddle region 311 extends distally from the elongate body. The paddle region includes a lateral side and two surface electrodes 313, 313′ that extend in parallel along the lateral side. The device shown also includes an articulating region 309 coupling the elongate body to the paddle region. The articulating region in this example is configured to articulate the paddle region in a plane extending through the elongate body (e.g., “up” and “down” in FIG. 3A). In FIG. 3A the paddle region is shown articulated up. As mentioned, the device may include a handle (not shown) at the proximal end that includes a control (e.g., an articulation control) configured to articulate the distal paddle in the plane.

FIG. 3B shows an example of the lateral side 314 of the paddle region 311, including the first 313 and second 313′ surface electrodes. As mentioned above, these surface electrodes may be configured as shown in FIG. 2A-2H, for example, each may include a semi-conducive layer, rounded edges and in some examples an air gap between the arc mitigating layer and the electrode surface.

FIGS. 3C, 3D and 3E show the example of the apparatus of FIGS. 3A-3B in a side view articulating through approximately 200 degrees in a plane (in this case the plane is the plane of the sheet). In FIG. 3C the apparatus 300 is articulated up approximately 100 degrees; this configuration may be used to drive the lateral side of the paddle region against a tissue that is in front of the device. In FIG. 3D the apparatus is shown straight (e.g., for insertion into an anatomical structure or tissue). In the straight configuration the apparatus 300 may be inserted laparoscopically through a standard port. Once inside of the body the apparatus can be articulated and/or rotated to position the surface electrodes against the target tissue. The electrodes may be driven against a tissue located to the side of the device. In FIG. 3E the apparatus 300 is shown articulated down approximately 100 degrees. This configuration may be used to treat a tissue proximal to the distal end of the device, e.g., facing the lateral side.

The elongate body may be generally a cannula, through which one or more channels may be included, including a working channel, a scope channel or the like. The elongate body may also secure and guide the articulation tendons (not shown) for articulating the hinged joints, as well as the electrical wire(s) for coupling to the electrodes. In the example shown in FIGS. 3A-3E the wiring (e.g., electrically coupling the surface electrodes to the connector at the proximal end) are not visible, nor are the articulation cables and/or a vacuum line or tubing.

The apparatus shown may have a diameter (e.g., transverse to the long axis) of between about 2 mm and about 15 mm; in the example shown in FIGS. 3A-3E the diameter is approximately 4 mm. The apparatus shown may include one or more vacuum ports on the lateral side of the device, e.g., adjacent to the surface electrodes, through which suction may be applied to hold the electrode surface to the tissue, and/or to remove air around the electrodes. Alternatively, the apparatus may be used without applying vacuum, simply by pressing the device against the tissue.

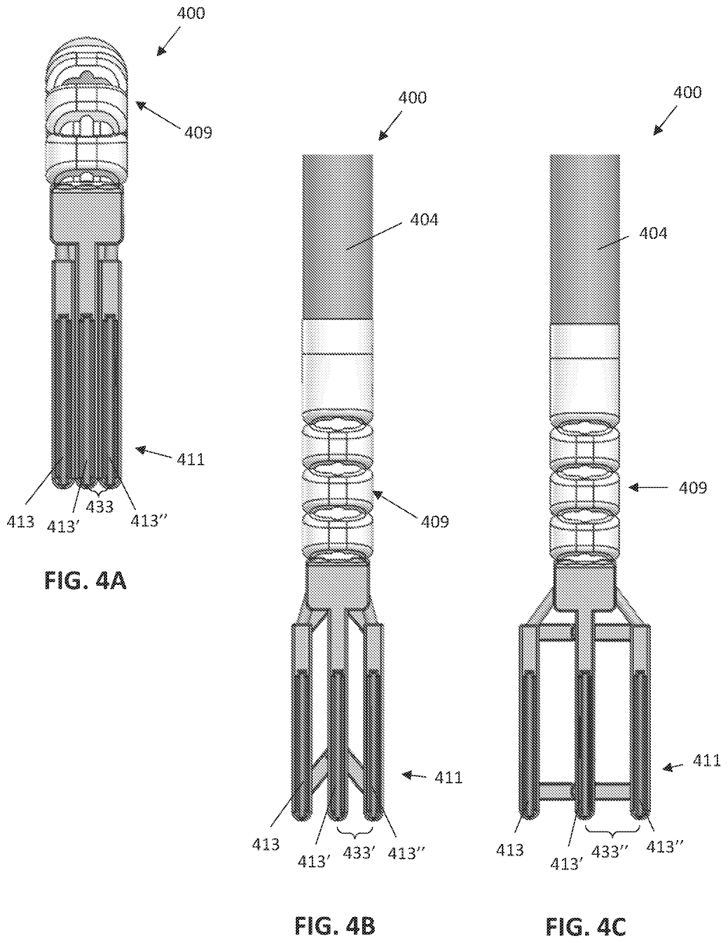

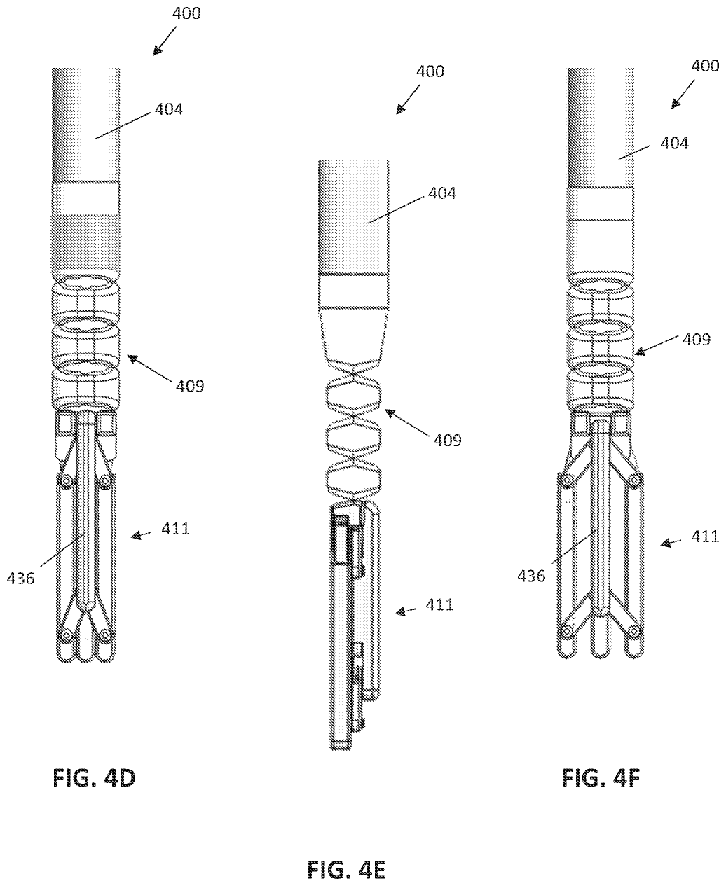

FIGS. 4A-4C illustrate another example of an apparatus 400 including a paddle region 411 that is coupled to an elongate body (e.g., cannula 404) through an articulating region 409. The articulating region (e.g., articulating joint section) may include one or more internal cables (not shown) and tubing (not shown). In the example shown in FIGS. 4A-4C the apparatus includes three surface electrodes 413, 413′, 413″ on the paddle region 411. In this configuration the paddle region is configured to expand laterally outwards, away from the long axis of the apparatus, to increase the separation between the surface electrodes. In FIG. 4A the apparatus is shown with the paddle in the unexpanded (e.g., collapsed) configuration that may allow delivery (e.g., insertion) through a port into the body, such as through a 10 mm port. The separation between the surface electrodes 433 is a minimum, and may be, e.g., about 2.5 mm. In FIG. 4B the apparatus may be deployed near the target tissue, e.g., by articulating the articulating region 409 so that the lateral side with the surface electrodes may be positioned adjacent to the target tissue. Once positioned, the paddle region may be expanded so that the surface electrodes are separated by a desired distance 433′ (e.g., in FIG. 4B the outer two electrodes are separated by approximately 11 mm. FIG. 4C shows the three-electrode design of FIGS. 4A-4B in a fully expanded configuration, with the maximum separation 433″ between the surface electrodes. For example in FIG. 4C the maximum separation may be about 10 mm between each electrode, with a 20 mm total separation between the outer electrodes. Any of the apparatuses described herein may include a sensor or gauge integrated into the apparatus to determine actual spacing of the electrodes, which may allow for precise voltage or power application and setting. In some examples, the apparatus may incorporate linear potentiometer. Also, in some implementations, the electrodes may comprise suction ports to apply vacuum, for example, around the electrodes.

FIGS. 4D-4F illustrate an example of the back side (FIGS. 4D and 4F) and side (FIG. 4E) of the apparatus of FIGS. 4A-4C, illustrating one mechanism for expanding and contracting the paddle region. For example FIG. 4D shows a rear view of the distal end region of the apparatus including the paddle region 411; in FIG. 4D the paddle region is shown in the collapsed (unexpanded) configuration. This configuration may be easily inserted through the port into the body. The paddle region includes a hinged frame that may be expanded outwards when the central member 436 is advanced distally, as shown in FIG. 4F, separating the outer surface electrodes. The central member may be driven distally or proximally by a cable or rod, which may be coupled to a handle in order to adjust the electrode separation. FIG. 4E shows a side view of the apparatus of FIGS. 4A-4D. FIG. 4F shows a rear view of the partially expanded paddle region (similar to the front view shown in FIG. 4B).

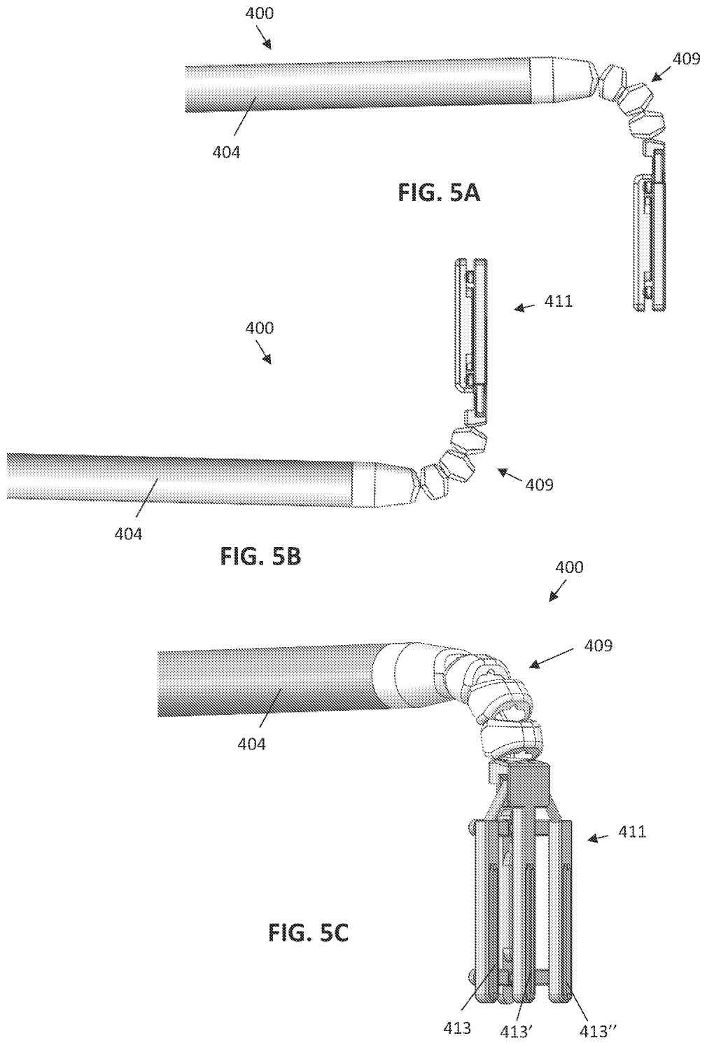

FIGS. 5A-5C show different views of the apparatus 400 of FIGS. 4A-4F with the paddle region 411 including three non-penetrating (e.g., surface) electrodes 413, 413′, 413″ whose spacing may be adjusted. In FIG. 5A the paddle region 411 is shown articulated 90 degrees “down” and in FIG. 5B the paddle region 411 is shown articulated 90 degrees “up”.

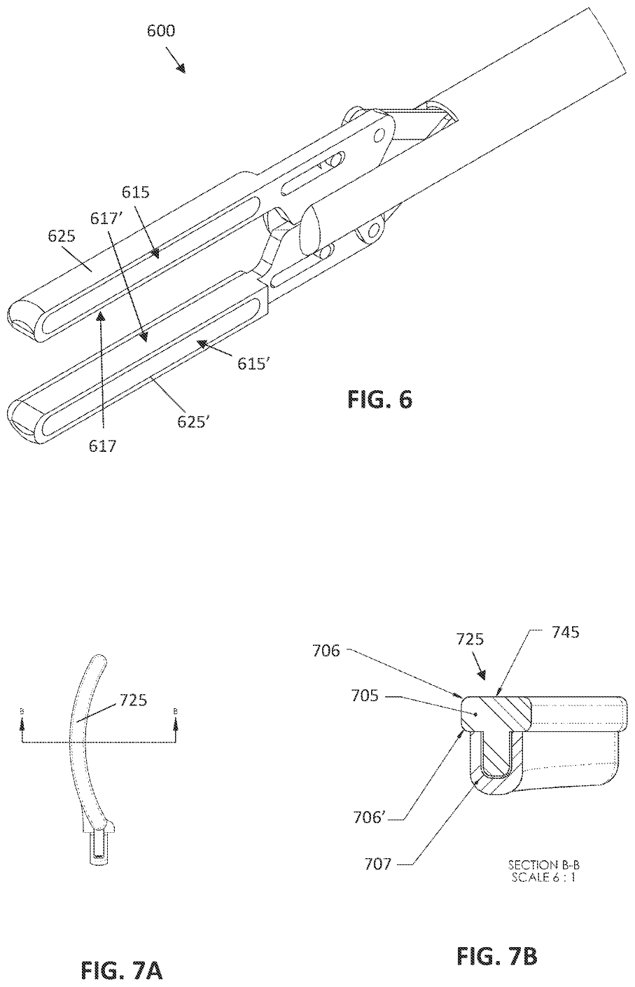

FIG. 6 shows another example of an apparatus 600 described herein, including a pair of articulating jaws on each of which may be positioned one or more contact electrodes. In FIG. 6 the apparatus includes a first (e.g., an upper) arm 625 and a second (a lower) arm 625′ that are configured to be actuated so that they open and close in parallel, as shown. The arms may be also referred to as “jaws”. The example shown in FIG. 6 may include surface electrodes on a lateral side 615, 615′ of each arm so that electrodes are side-facing electrodes, and/or the electrodes may be positioned on the inner surfaces 617, 617′ so that tissue may be held between the arms and against the surface electrodes in a clamp-type configuration.

In the example shown in FIG. 6 with the electrodes on the surfaces 617 and 617′ the apparatus is configured to clamp onto the tissue so that the tissue is held between the arms or jaws. The force of the tissue being held in the jaws may both secure the tissue in position and may, in some examples, deflect the arc mitigating layer of the contact electrode against the electrode surface, as described in reference to FIG. 2H. In some implementations the electrodes may be positioned on both inner surfaces 617, 617′ and also on the lateral surfaces 615, 615′ and, in use, only the appropriate set of electrodes may be selectively activated as needed, for example, either for applying electrical treatment between the side facing electrodes or between the “clamping” electrodes depending on the user preference and/or the configuration and position of the target tissue. Thus any of these applicators may include a switch to multiplex between one or more sets of electrodes on the applicator.

FIGS. 7A and 7B illustrate alternative examples of arms or jaws that may be used with an apparatus of the present disclosure. In FIG. 7A the apparatus includes curved or bent jaws 725. FIG. 7B shows an example of a section through the curved jaw of FIG. 7A, showing that the electrode 705 may include a tissue contacting surface 745 that includes rounded corners/edges 706 (e.g., fillets) on tissue contacting surface 745 and also rounded edges 706′ on non-tissue contacting edges/surface. The rounded edges 706′ on the non-tissue contacting or facing edges may also reduce the peak electrical field and prevent arcing from this (e.g., lower) edge. Adding a rounded edge/corner can reduce the peak electric field, for example, by 15% to 30%, by 20% to 25%, depending on the type of the tissue. Any appropriate radius of the curvature for the rounded edge (fillet) may be used, for example, if the electrode has a thickness of t, the radius of curvature may be larger than about t/8 (e.g., larger than about t/7, larger than about t/6, larger than about t/5, larger than about t/4, larger than about t/3, between about t/8 and about 4t, between about t/8 and 2t, etc.). For example, in some apparatuses the radius of curvature of the curved edge (fillet) may be, e.g., between about 0.1 mm to 0.5 mm for certain dimensions of the electrodes. The electrode in this example is secured within an electrically insulating material 707 forming the base.

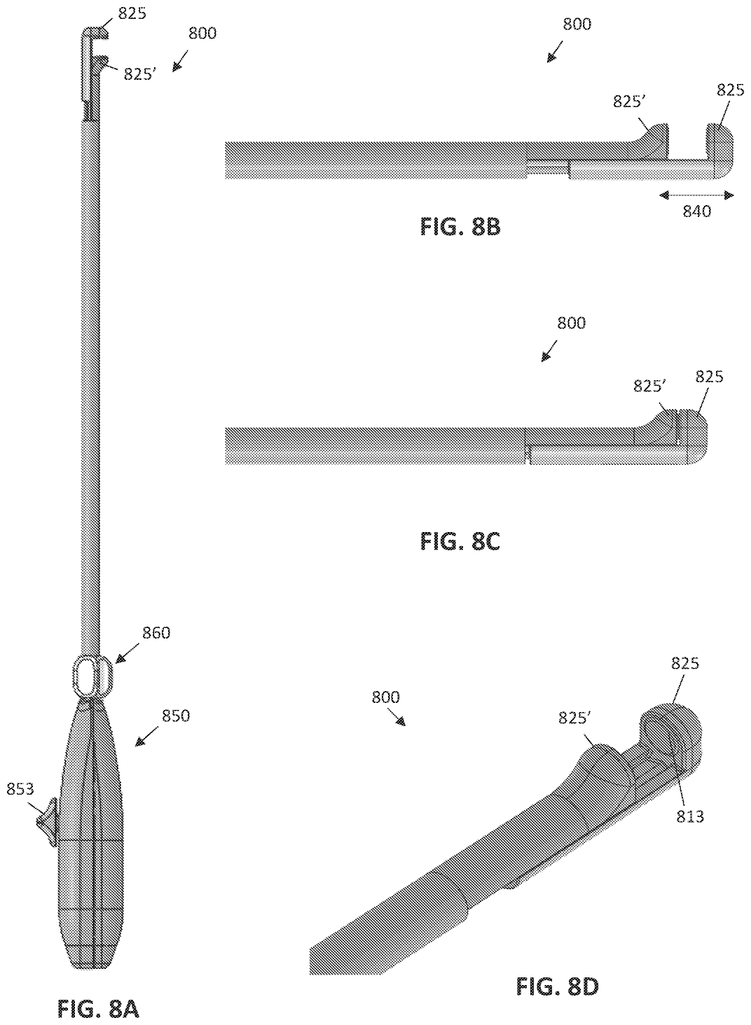

FIGS. 8A-8D illustrate another example of a clamping applicator apparatus 800. In this example the clamping apparatus may be configured for treating a tissue, such as the vocal cord/vocal fold, for example. The apparatus includes an upper (distal) clamping jaw 825 an a lower (proximal) jaw 825′. In this example the upper jaw 825 moves distally to proximally as shown by arrow 840 (FIG. 8B) in the longitudinal axis (long axis) of the device to adjust the jaw mouth opening into which tissue may be held. In different implementations, instead of the distal jaw, the proximal jaw may move relative to the distal jaw, or each of the jaws may be movable relative to each other. The contact electrodes 813 are illustrated positioned on the inside faces of the jaws 825, 825′ so as to contact the target tissue to be treated. The contact electrode 813 on the upper/distal clamping jaw is shown in FIG. 8D. FIGS. 8B and 8C illustrate the jaw of the apparatus in an open (FIG. 8B) and closed (FIG. 8C) configuration. In practice, the jaw may be clamped onto a tissue so as to apply treatment therebetween. In some examples the apparatus may include an articulation region (articulation joint) as described above. The elongate body may be configured as a cannula, as described in reference to FIGS. 3A-3E and 4A-4F. In some additional examples, various features of the apparatuses of FIG. 6 and/or FIGS. 7A-7B may be combined with and incorporated into the example of FIGS. 8A-D.

Any of these apparatuses may also include a rotation control (e.g., rotation knob) 860 that may be configured to allow the clamp to be rotated without rotating the handle 850 (e.g., just rotating the elongate body and/or the distal end clamp or paddle region). The handle 850 may also include a control 853 for actuating the clamping jaws and/or controlling clamping of the apparatus. In some example, the handle may comprise a plunger and/or a latch for actuating/controlling the clamping. The control 853 in FIG. 8A is shown as a clamping slide that may be used to incorporate a sensor or gauge to determine the spacing between the electrodes. In general, any of the apparatuses described herein may include a sensor or gauge configured to determine the spacing between the electrodes. The output of this sensor or gauge may be passed to a controller (e.g., a control 144 on or in the pulse generator (see, e.g., FIG. 1) and, in some implementations, the output of the sensor may allow a system to automatically setup treatment parameters based on the spacing between the jaws and/or electrodes. In some examples, the apparatus may incorporate linear potentiometer. In variations in which the apparatus includes an articulation joint, the apparatus may further include an articulation control (not shown).

FIG. 8B shows a side view of the jaw assembly of the apparatus 800. In this example the jaws can be opened (e.g., up to 25 mm or more, as needed). As mentioned, the apparatus may include an articulation joint similar to that shown in FIGS. 3A-3E. Although the examples described herein may include contact or surface electrodes, in some implementations these apparatuses may be configured for use with needle electrodes, wire electrodes, or the like.

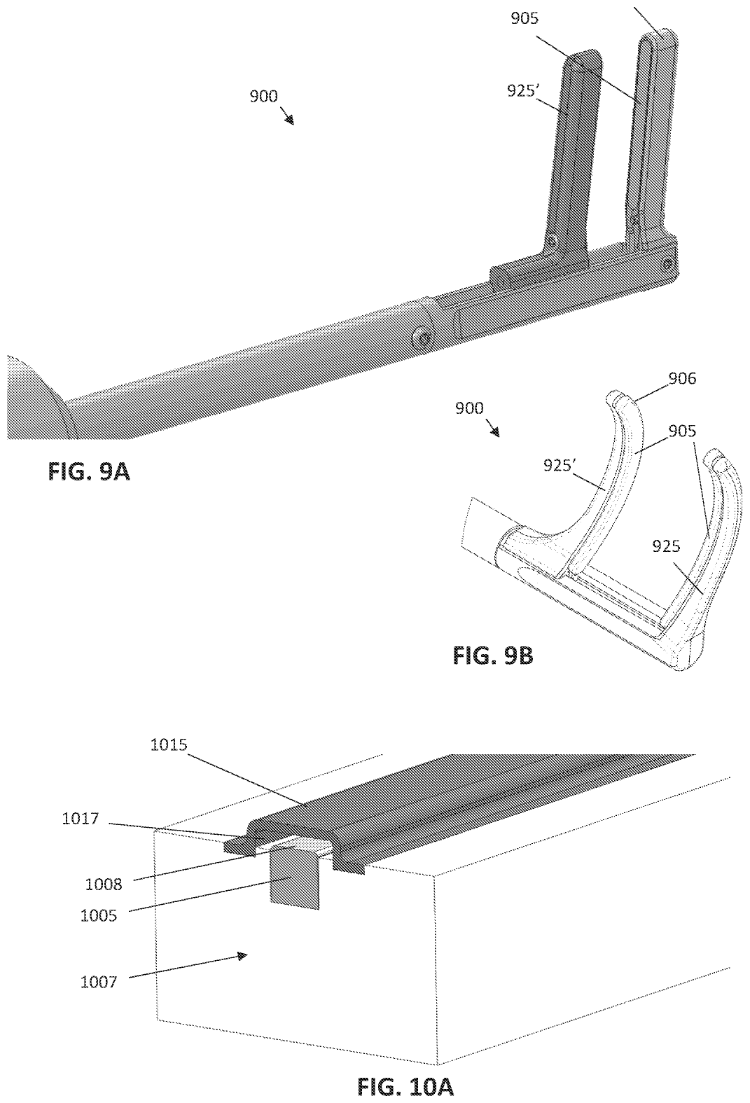

FIG. 9A shows another example of a clamping apparatus 900 including a pair of jaws (an upper or distal jaw 925 and a lower or proximal jaw 925′). The apparatus in this example includes a first contact electrode 905 on the inner side of the distal jaw and the second electrode on opposite side of the proximal jaw (not visible in FIG. 9). The opening diameter of the jaws may be adjusted, e.g., by a control on the handle, to move, for example, the proximal jaw proximally or distally, or alternatively, to move the distal jaw axially relative to the proximal jaw.

FIG. 9B shows another example of a clamping apparatus 900 having substantially parallel jaws positioned at an angle (for example, 90 degrees/perpendicular) to the shaft of the apparatus but with a distal and proximal jaws 925, 925′ of the clamping apparatus shaped similar to the bent or curved jaws 725 of FIG. 7A. Also, the electrodes 905 of FIG. 9B have rounded edges/corners 906 (similar to the rounded edges 706, 706′ of FIG. 7) to reduce or prevent arcing as explained in reference to FIG. 7B. In this example, there are rounded corners on all electrode edges, however, in some implementations the rounded corners/edges may be just on or around a tissue contacting surfaces of the electrode. The electrodes 905 in this example are positioned for clamping tissue therebetween, so they are positioned on the inner sides of each respective jaw and project from the respective jaws 925, 925′ inwards (for example, by 1 mm, 2 mm, 3 mm) as seen in FIG. 9B. Similar to FIG. 7B, the electrodes 905 may be secured within an electrically insulating material forming the base of each jaw. The spacing between the jaws, and therefore between the electrodes, may be adjusted, for example, by a control on the handle, to move, for example, the proximal jaw proximally or distally. For example, a proximal jaw may be spring loaded. Like with the other examples, the jaws 925, 925′ may include surface electrodes on a lateral side of each jaw (not shown) so that such electrodes on the lateral side are side facing electrodes (similar to those shown in FIG. 6). Such side facing electrodes may be, for example, in addition to the inner tissue clamping electrodes 905, and the apparatus 900 may comprise a control of a spacing between the side facing electrodes in addition to the control of the spacing between the clamping electrodes. Also, like with the other examples, the apparatus may be configured to detect spacing between the electrodes and send such spacing information to the control of the pulse generator system to adjust one or more parameters of the pulses. Examples of FIGS. 9A-9B may be especially useful for treatment of cardiac arrhythmia, e.g., atrial fibrillation. For example, a slightly curved or bent configuration of the jaws may assist in isolation of the right and left pulmonary veins and creating other atrial lesions for preventing atrial fibrillation. The apparatuses of the present disclosure may provide a faster, more effective and robust procedure to treat atrial fibrillation and such procedure may be performed with a single apparatus.

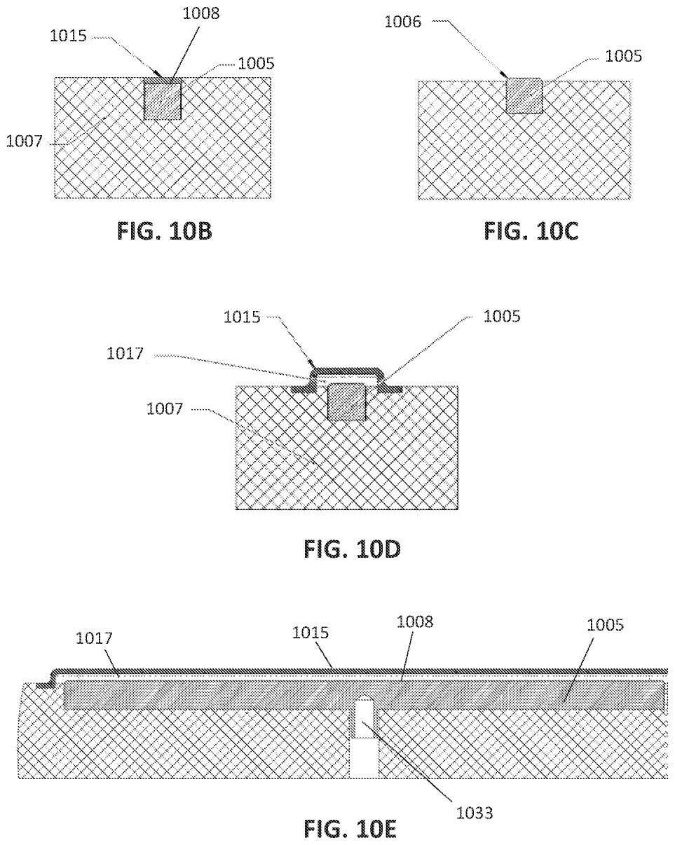

As mentioned above, the applicators with the contact electrodes of the present disclosure may be configured to reduce or prevent arcing. FIG. 10A shows one example of a contact electrode with additional arc mitigating features. In FIG. 10A the contact electrode 1005 includes an electrode surface 1008 (exposed or open surface of the electrode). The electrode 1005 is supported, for example, on all sides other than the electrode surface 1008 by an electrical insulative material 1007. A flexible arc mitigating layer 1015 is positioned over the electrode surface 1008 with a gap region 1017 between the arc mitigating layer and the electrode surface. FIGS. 10B-10E show alternative configurations of the contact electrode. In FIG. 10B the contact electrode (e.g., a stainless steel electrode) 1005 is supported (and partially surrounded) by an insulative support 1007. The outer surface 1008 of the electrode 1005 is covered by an arc mitigating layer 1015. FIG. 10C shows an example of a contact electrode 1005 that includes rounded edges 1006 (e.g., fillets) on the outer or exposed electrode surface 1008 to prevent arcing. FIG. 10D shows an example of a section through a contact electrode similar to that shown in FIG. 10A, including an electrode surface 1008 on an electrode 1005 that is separated from an arc mitigating layer 1015 by an air gap 1017. FIG. 10E shows an alternative section, e.g., longitudinal section, through the electrode of FIG. 10D, and shows an electrical connection 1033 that may be coupled to a wire or conducive material for electrically coupling to the pulse generator.

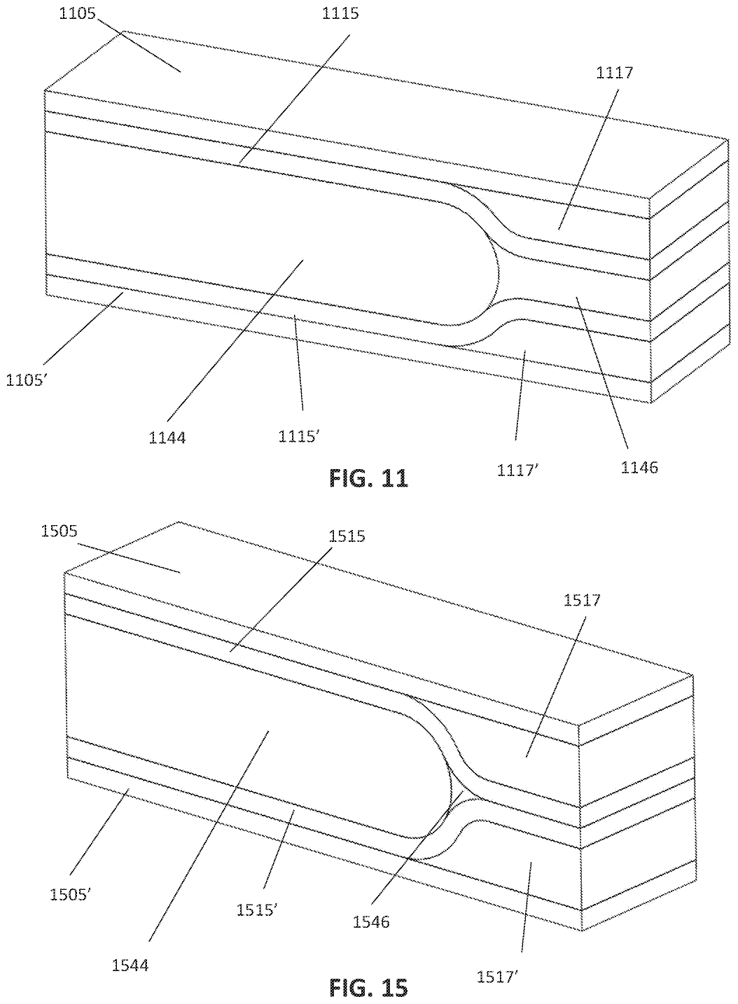

FIG. 11 illustrates one example of a model of a parallel jaw having two contact electrodes similar to those shown in FIGS. 9 and 10A, showing an exemplary section through the jaw when clamping on a target tissue 1144 (e.g., cardiac tissue in this model). The geometry shown in FIG. 11 was used to model electric fields as described below and showed a significant reduction in arcing likelihood based on the resulting distribution of the electric field. In FIG. 11, the model includes a first electrode 1105 and a second electrode 1105′. The first electrode 1105 and the second electrode 1105′ each is covered, respectively, by an arc mitigating layer 1115, 1115′ that is normally spaced apart from the electrode surface by a respective air gaps 1117, 1117′. In FIG. 11, the jaws are clamped onto a sample tissue 1144 so that the arc mitigating layer 1115, 1115′ is driven against the electrode surface where the tissue is clamped, while the air gap 1117, 1117′ remains over the unclamped regions. In the example shown in FIG. 11 the jaws are closed over the tissue so that the opposite semi-conducive layers 1115, 1115′ (including in a region that is not in contact with the target tissue) do not contact each other but are separated by blood or air 1146.

The apparatuses described herein, including the clamping applicators such as those shown in FIGS. 8A, 9B and 11, may be used to treat a cardiac tissue such as the pulmonary veins. For example, these apparatuses may be used as part of a procedure for bilateral pulmonary vein isolation (without atriotomy) during a minimally invasive aortic valve replacement or during Maze procedure to treat atrial fibrillation. The right pulmonary veins may be ablated by applying sub-microsecond pulsing as described herein, from a clamp such as the apparatus shown, for example, in FIG. 9B or 11. The apparatus may be introduced into the oblique sinus under the aorta, to ablate the left pulmonary veins. These devices provide numerous benefits in cardiac applications, as stated above. Moreover, the use of sub-microsecond pulsed electric fields in combination with the configurations of the apparatuses of the present disclosure allows to safely treat (e.g. ablate) close to vessels and valves.

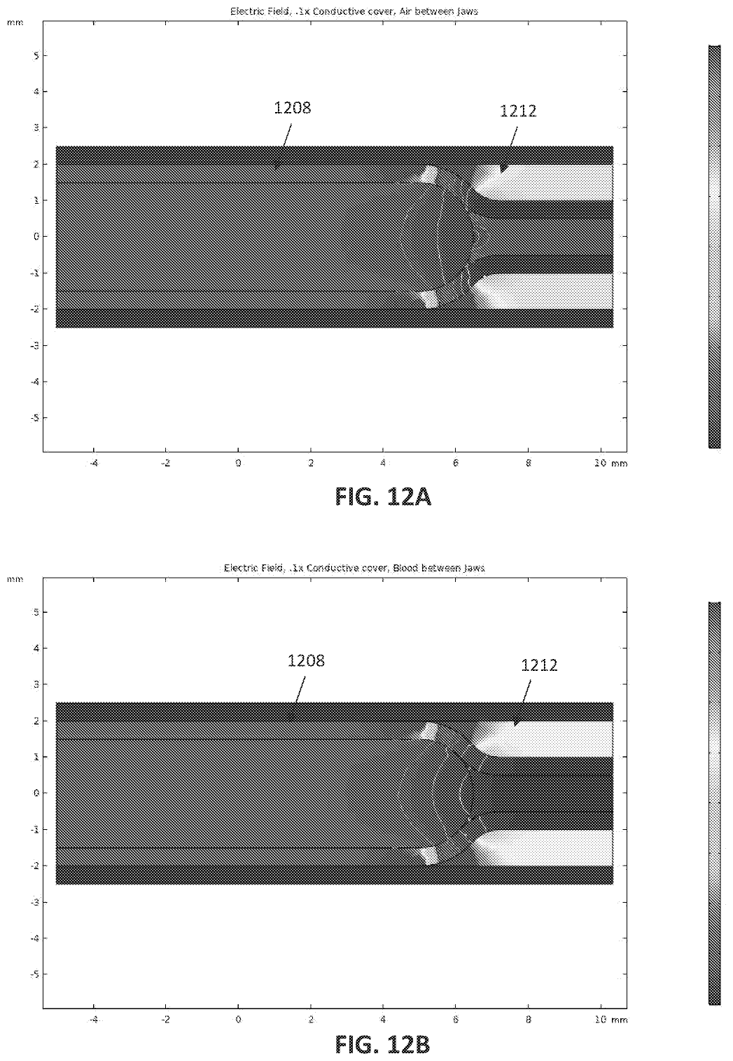

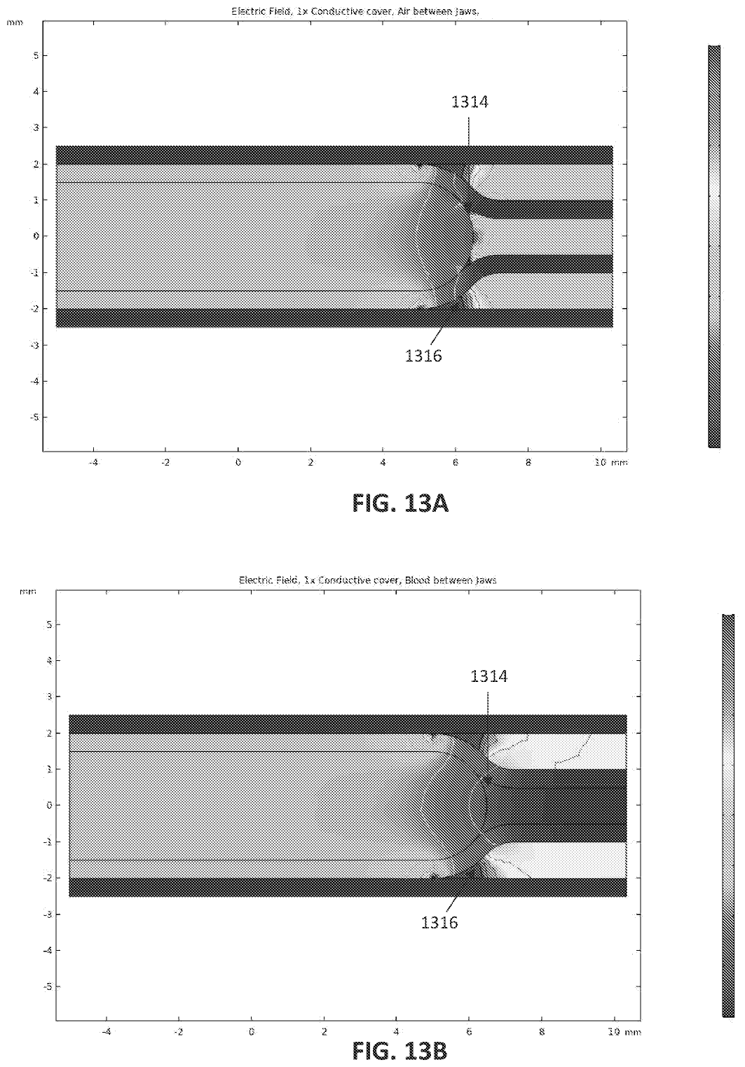

FIGS. 12A-12B, 13A-13B and 14A-14C shows examples of simulated electrical fields when applying high voltage energy, such as sub-microsecond pulses, between the jaws of the applicator tool of the present disclosure. In FIGS. 12A-12B the electric field is simulated with the arc mitigating layers having a conductivity that is approximately 0.1 time the conductivity of the target tissue (e.g., cardiac tissue), when the applied field with either air between the jaws (FIG. 12A) or blood between the jaws (FIG. 12B). As shown, the resulting field is highest over the region of highest contact 1208 between the arc mitigating layer and the tissue (where the arc mitigating layer displaces the air gap) and falls off rapidly where the air gap remains 1212. In FIGS. 13A-13B the electric field is simulated with the arc mitigating layers having a conductivity that is approximately the same as the conductivity of the target tissue (e.g., cardiac tissue), with either air between the jaws (FIG. 13A) or blood between the jaws (FIG. 13B). The simulation shows a region of high electric field at the space 1316 between the electrode and the inside of the arc mitigation layer of the tissue in both cases, however as this region is internal to the air gap between the arc mitigating layer and the electrode surface, there is no risk of arcing. In FIG. 13A the small separation 1314 between the arc mitigating layer and the tissue also shows a larger field in air, but not in blood (FIG. 13B).

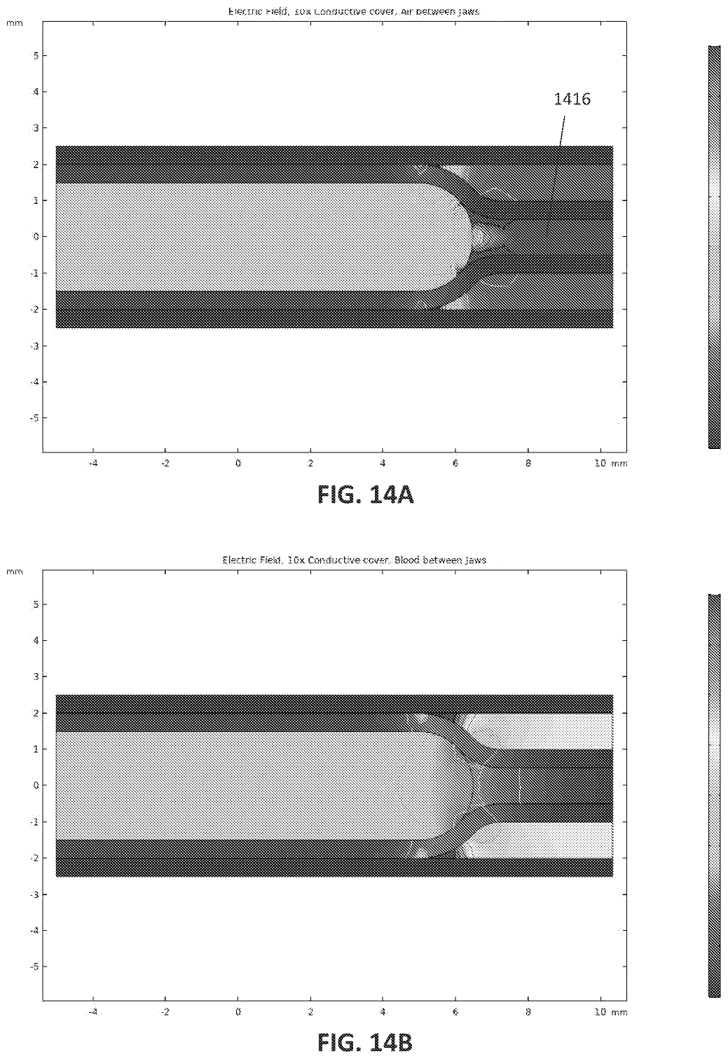

In FIGS. 14A-14B the electric field is simulated with the arc mitigating layers having a conductivity that is approximately 10 times the conductivity of the target tissue (e.g., cardiac tissue), when the applied field is 5 kV with either air between the jaws (FIG. 14A) or blood between the jaws (FIG. 14B). The simulation shows a significant electrical field in a region 1416 between the non-tissue containing jaws when there is air (but not blood) between the jaws. Any of the apparatuses described herein may be configured to minimize or prevent this gap region and in particular any gap between the tissue and the arc mitigation layer.

FIG. 15 shows a model of a parallel jaw having two contact electrodes similar to that shown in FIG. 11, but in FIG. 15 when the jaws are closed the arc mitigating layers that are not in contact with tissue are in contact with each other (except for a small transition area 1546). The apparatus may be configured to minimize or eliminate the transition area 1546 so that the tissue is completely or nearly completely contacted by the arc mitigation layer. For example, the arc mitigation layer may be formed of a compliant (e.g., highly flexible material) and/or may have a thickness that permits it to be more highly compliant. The gap region 1517 may be larger (e.g., more than half the size of the uncompressed thickness of the tissue, more than 55%, more than 60%, more than 70%, more than 80%, etc.) to allow a wider thickness of tissues to be clamped therebetween.

In FIG. 15, the model includes a first electrode 1505 and a second electrode 1505′. The first electrode 1505 and the second electrode 1505′ each is covered, respectively, by an arc mitigation layer 1515, 1515′ that is normally spaced apart from the electrode surface by a respective air gaps 1517, 1517′. In FIG. 15, the jaws are clamped onto a sample tissue 1544 so that each arc mitigating layer 1505, 1505′ is driven against the electrode surface where the tissue is clamped, while the air gap 1517, 1517′ remains only over the unclamped regions. In the example shown in FIG. 15 the jaws are closed over the tissue such that the opposite semi-conducive layers 1515, 1515′ in a region that is not in contact with the target tissue contact each other, except for a very small transition gap 1546. As mentioned, the apparatus may be configured to minimize or eliminate this transition gap 1546.

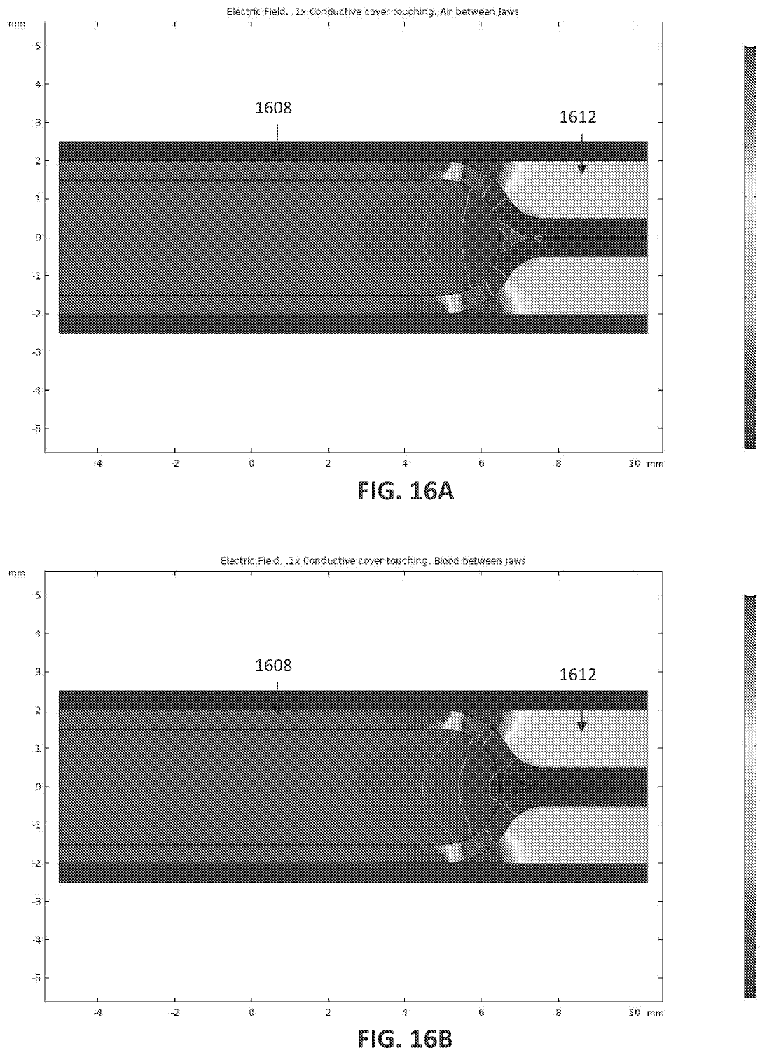

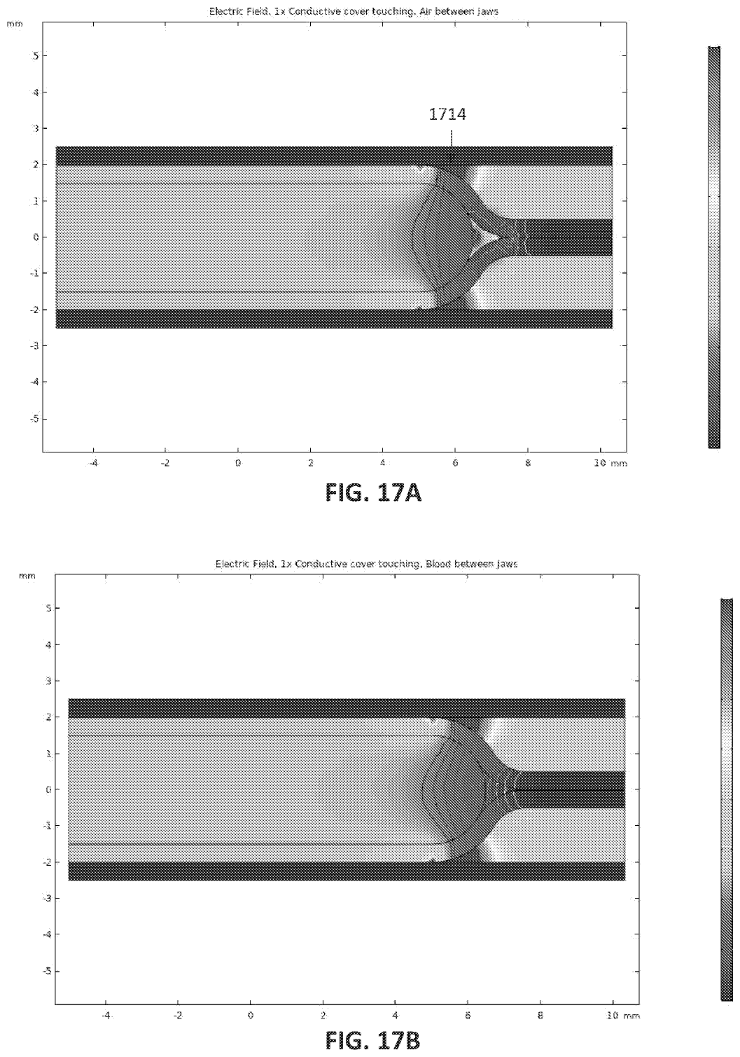

FIGS. 16A-16B, 17A-17B and 18A-18B show the resulting simulations of the electrical fields based on the applicator configuration of FIG. 15. In FIG. 16A-16B the electric field is simulated with the arc mitigating layers having a conductivity that is approximately 0.1 time the conductivity of the target tissue (e.g., cardiac tissue), with either air between the jaws (FIG. 16A) or blood between the jaws (FIG. 16B). As shown, the resulting field is highest over the region of highest contact 1608 and falls off rapidly where the air gap remains 1612. In FIGS. 17A-17B the electric field is simulated with the arc mitigating layers have a conductivity that is approximately the same as the conductivity of the target tissue (e.g., cardiac tissue), with either air between the jaws (FIG. 17A) or blood between the jaws (FIG. 17B). As in FIGS. 13A-13B (in relation to the model of FIG. 11), the simulation shows a region 1714 of high electric field within the air gap of the electrode assembly in both cases, but this internal region cannot result in arcing.

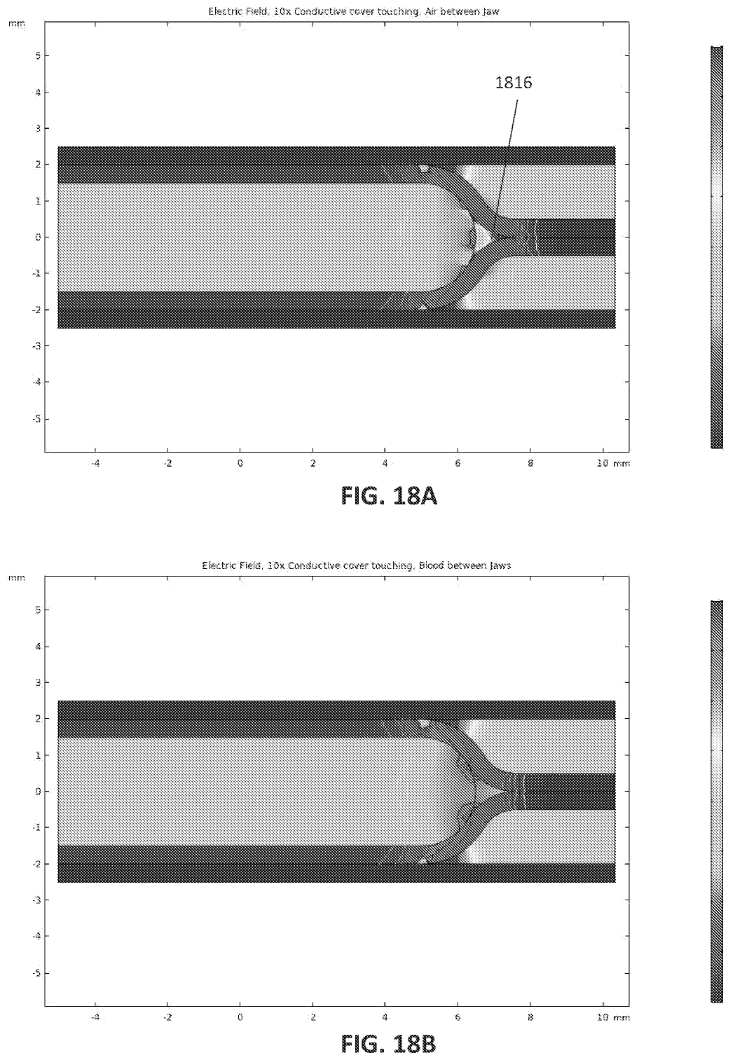

In FIGS. 18A-18B the electric field is simulated with the arc mitigating layers having a conductivity that is approximately 10 times the conductivity of the target tissue (e.g., cardiac tissue) with either air between the jaws (FIG. 18A) or blood between the jaws (FIG. 18B). The simulation shows a significant electrical field 1816 between the small region of non-contact between the arc mitigating layers when there is air (but not blood) between the jaws.

The simulation data shown in FIGS. 11-18B suggests that it may be beneficial to select the conductance of the arc mitigating layer (e.g., intermediate conductivity layer) so that it is within one order of magnitude of the conductance of the expected conductance of the tissue to be treated. For example, in any of these apparatuses the arc mitigation layer may have a conductance that is between 0.1 times the expected tissue conductance and 10 times the expected tissue conductance (e.g., less than 10 times the expected tissue conductance, less than 5 times the expected tissue conductance, less than 3 times the expected tissue conductance, less than 2 times the expected tissue conductance, approximately the same as the expected tissue conductance, less than the expected tissue conductance, less than 0.9 times the expected tissue conductance, less than 0.8 times the expected tissue conductance, less than 0.7 times the expected tissue conductance, less than 0.6 times the expected tissue conductance, less than 0.5 times the expected tissue conductance, less than 0.4 times the expected tissue conductance, less than 0.3 times the expected tissue conductance, less than 0.2 times the expected tissue conductance, less than 0.1 times the expected tissue conductance, less than 0.09 times the expected tissue conductance, etc.).

Experiments using the applicator apparatuses described herein show that all of these configurations resulted in robust treatment of the sample tissue. Similar experiments showed the occasional arcing of exposed electrodes that did not include an arc mitigation layer was completely eliminated with the use of an arc mitigating electrode, both with and without a gap between the arc mitigating electrode and the electrode surface.

### Methods of Treatment

As mentioned above, any of these apparatuses may be used to treat a tissue and are particularly useful when at least a portion of the electrode may be exposed to air or blood during treatment. In particular, these methods may be used as part of a laparoscopic procedure to treat a tissue.

For example, an apparatus as described herein having non-penetrating (e.g., surface) electrodes may be used to contact tissue of the female urogenital or reproductive system (e.g., ovaries, fallopian tubes and the tissue lining the pelvis). For example, an apparatus may include a paddle region including electrodes as described herein and may be used to treat endometriosis. The apparatus may be inserted (and in some examples expanded out) into the body and pushed against the tissue affected by endometriosis. In some examples suction may be used to help secure the surface electrodes to the target tissue.

In some examples the surface electrodes as described herein may be used to contact and treat different conditions, for example, one or more lesions (e.g., growths, such as nodules, polyps and/or cysts) on a vocal fold (e.g., vocal cord). For example, an apparatus configured as a paddle region including surface electrodes as described herein may be inserted into the patient (e.g., through the mouth or via an incision) to approach the vocal cords/vocal folds. In some examples the paddle region may be expanded out and may be pushed against tissue of the vocal fold to treat a vocal cord lesion. In some examples the target tissue (vocal cords/vocal folds) may be clamped between two or more surface electrodes as described. One or more treatments may be applied. These methods may also be used to treat laryngeal papilloma among other dermatological lesions and conditions.

The apparatuses as described herein may be also used to treat cardiac tissue. For example an applicator as described herein may be inserted through the vasculature and used to clamp or otherwise be secured against a cardiac tissue, for example, to treat atrial fibrillation among other cardiac conditions.

Other indications may include treatment of dermatologic lesions, internal tumors in an organ, treatment of vessels, including blood vessels, ducts, intestines, treatment of eye lids, lips, tongue, ureter, urethra, gallbladder, ducts, bile duct, lymph nodes, rectum, esophagus, heart, liver, intestine, stomach, pancreas, lung, uterus, fallopian tube, fingers, ear, nose, any vessel, spleen, kidney, etc. For example, the clamping apparatuses described herein may be used, for example, to treat any tissue that can fit between the jaws.

In particular, these apparatuses may be particularly well suited for treating indications in which larger treatment areas are desired, so that the surface electrode may be made relatively long without worrying about arcing, particularly when the electrode is configured to include rounded edges (e.g., fillets) and/or an arc mitigating layer, as described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

All publications and patent applications mentioned in this specification are herein incorporated by reference in their entirety to the same extent as if each individual publication or patent application was specifically and individually indicated to be incorporated by reference.

1.-40. (canceled)

41. An electrode apparatus configured to limit arcing, the apparatus comprising:
a first electrically insulative support;
a first electrode supported by the first electrically insulative support, the first electrode comprising a first electrode surface; and
a second electrode supported by the first electrically insulative support or a second electrically insulative support, the second electrode comprising a second electrode surface,
wherein the first electrode and the second electrode are configured to pass electrical pulses having an amplitude of at least 0.1 kV therebetween and
wherein the apparatus further comprises a first arc mitigating layer configured to reduce or eliminate arcing between the first electrode and the second electrode when passing the electrical pulses, the first arc mitigating layer being formed of material having a conductivity that is less than the conductivity of the first electrode surface.

42. The apparatus of claim 41, wherein the material has a conductivity that is less than, approximately equal to or greater than a conductivity of a target tissue.

43. The apparatus of claim 41, wherein the arc mitigating layer is formed of a polymeric material that is doped or otherwise treated to a have a conductivity within a target range.

44. The apparatus of claim 43, wherein the polymeric material comprises a silicone polymer and a doping material comprises a carbon.

45. The apparatus of claim 41, wherein the first and the second electrodes each has a length larger than a width.

46. The apparatus of claim 41, wherein the first arc mitigating layer comprises a flexible membrane or a coating.

47. The apparatus of claim 41, wherein the first electrode and the second electrode are surface or plate electrodes.

48. The apparatus of claim 41, wherein the first electrode and the second electrode are separated by a fixed distance.

49. The apparatus of claim 41, wherein the first electrode is positioned on a first side of a first jaw and the second electrode is positioned on a second side of a second jaw of the apparatus, wherein the first side of the first jaw and the second side of the second jaw are configured to secure a tissue to be treated therebetween.

50. The apparatus of claim 41, wherein the first electrode is on a lateral side of a first jaw and the second electrode is on a lateral side of a second jaw, wherein the lateral side of the first jaw is in a same plane as the lateral side of the second jaw.

51. The apparatus of claim 49, wherein the first jaw and the second jaw are configured to open and close so that the first electrode and the second electrode remain parallel to each other.

52. The apparatus of claim 49, wherein the first jaw and the second jaw are configured to move axially relative to each other.

53. The apparatus of claim 41, wherein the arc mitigating layer is secured to, attached over, at least partially covering or at least partially separated from the first electrode surface.

54. The apparatus of claim 53, the apparatus comprising a first gap region between the first arc mitigating layer and the first electrode surface in an unactuated state.

55. The apparatus of claim 54, wherein the first gap region comprises an air gap.

56. The apparatus of claim 54, wherein the first gap region is 0.5 mm or larger.

57. The apparatus of claim 41, wherein the first arc mitigating layer is configured to be deflected against the first electrode surface when force is applied against the first arc mitigating layer.

58. The apparatus of claim 41, wherein the first electrode and the second electrode each comprises rounded edges.

59. The apparatus of claim 41, wherein the first arc mitigating layer has a conductivity that is between 0.01 S/m and 10 S/m.

60. The apparatus of claim 41, further comprising one or more suction ports configured to apply suction to draw a tissue against at least one or both of the first electrode and the second electrode.

61. The apparatus of claim 41, wherein the apparatus comprises a paddle region extending distally from an elongate body and wherein the first and the second electrodes are on the paddle region.

62. The apparatus of claim 61, further comprising an articulating region coupling the elongate body to the paddle region, wherein the articulating region is configured to articulate the paddle region in a plane extending through the elongate body.

63. The apparatus of claim 61, wherein the paddle region is configured to expand in width to increase spacing between the first and the second electrodes.

64. An electrode apparatus configured to limit arcing, the apparatus comprising:
a first jaw and a second jaw configured to open and close to secure a tissue to be treated therebetween;
a first electrode supported by the first jaw; and
a second electrode supported by the second jaw,
wherein the first and the second electrode are configured to pass electrical pulses having an amplitude of at least 0.1 kV therebetween and
wherein the apparatus further comprises a first arc mitigating layer and a second arc mitigating layer configured to reduce or eliminate arcing between the first electrode and the second electrode when passing the electrical pulses, the first and the second arc mitigating layers being formed of material having a conductivity that is less than the conductivity of the respective first and second electrodes.
